(19) United States
(12) Patent Application Publication
(10) Pub. No.: US 2022/0087952 A1
(43) Pub. Date: Mar. 24, 2022

(54) ANTI-APICOMPLEXAN COMPOSITIONS

(71) Applicants: THE STATE OF ISRAEL, MINISTRY OF AGRICULTURE & RURAL DEVELOPMENT, AGRICULTURAL RESEARCH ORGANIZATION, Rishon Lezion (IL); THE NATIONAL SOCIETY FOR HEALTH RESEARCH & SERVICES (R.A.), Shefa Amr (IL)

(72) Inventors: Hassan AZAIZEH, Dabburia (IL); Serge LANDAU, Caesarea (IL); Liora GUR, Ramat-Yishay (IL)

(73) Assignees: THE STATE OF ISRAEL, MINISTRY OF AGRICULTURE & RURAL DEVELOPMENT, AGRICULTURAL RESEARCH ORGANIZATION, Rishon Lezion (IL); THE NATIONAL SOCIETY FOR HEALTH RESEARCH & SERVICES (R.A.), Shefa Amr (IL)

(21) Appl. No.: 17/424,527

(22) PCT Filed: Jan. 19, 2020
(86) PCT No.: PCT/IL2020/050078

§ 371 Date: Jul. 21, 2021

Related U.S. Application Data
(60) Provisional application No. 62/794,731, filed on Jan. 21, 2019.
(60) Provisional application No. 62/880,101, filed on Jul. 30, 2019.

(51) Int. Cl. A61K 31/05; A61K 31/192; A61K 31/7048; A61K 36/63; A61P 33/00

(52) U.S. Cl. CPC A61K 31/05; A61K 31/192; A61K 31/7048; A61K 36/63; A61K 2236/39; A61K 2236/331; A61K 2236/51; A61K 2236/53; A61P 33/00

(57) ABSTRACT

The invention is directed to Anti-Apicomplexan compositions, methods for producing the same, and methods for their use.

### FIELD OF THE INVENTION

The present invention is directed to Anti-Apicomplexan compositions, methods of preparing the same and methods of administering the same to animals, for example poultry, for preventing and treating infection and disease.

### BACKGROUND OF THE INVENTION

Apicomplexa protozoa, in particular, Eimeriorina and Cryptosporidiidae, are parasitic to livestock. Cryptosporidia spp. include 30 named species and cause intestinal infections in vertebrates, including Man. Coccidiosis is a widespread disease characterized by diarrhea, emaciation, impaired feed intake, as well as lack of feed efficiency and growth and even mortality in a wide array of organisms, ranging from poultry to ruminants and lagomorphs. Coccidiosis is known to be caused by the Apicomplexan parasite Eimeria spp. The life cycle of Eimeria spp. involves only one host, but encompasses two major stages. The first stage includes a free oocyst, the source of which may be animal feces. The free oocyst thrives particularly in litter in the premises of animals and is known to sporulate under suitable temperature and humidity conditions. The second stage includes a sequence of developmental, a-sexual and sexual stages, which thrive in the epithelia of host gastro-intestinal tract and are eventually defecated in the form of unsporulated oocysts.

Poultry is known to be affected by seven different species of Eimeria. The Eimeria tenella species, involved in the coccidiosis of broiler chicks from week 1 to week 8, causing, e.g., caecum lesions, is known to be one of the most harmful species. Further, the E. necatrix species, known to affect chicks from weeks 5-7, by, e.g., causing lesions in the medial jejunum and the caeca, is also considered to be highly harmful. In addition, for example, the E. acervulina species affects young layers and features lesions in the duodenum and proximal jejunum. In infected surroundings, such as poultry enclosures, the parasite may be present on the floor in the form of oocysts that are ingested by chicks. Oocysts are highly resilient to adverse conditions and therefore are extremely difficult to treat. Oocysts become infective once sporulated, a process that requires 25-30° C. and oxygen for 2-4 days, which is a condition met in all poultry enclosures used in the industry since the poultry itself requires such conditions.

Generally, coccidiosis is treated by vaccination as well as by administering coccidiostats, i.e., substances that control the proliferation of Eimeria spp. Each coccidiostat is known to impair a certain metabolic cycle in the parasite, for example, ethopabate and sulphonamides/amprolium impair the metabolism of folic acid and thiamin, respectively; and decoquinate may impair mitochondrial activity. Coccidiostats may be Eimeria species-specific and therefore, ethopabate and sulphonamide may be used to specifically combat E. acervulina E. maxima, and E. brunetti. Some coccidiostats may be stage-specific, for example, amprolium may affect the first schizont generation, sulphonamide may affect the second schizont stage and clazuril, diclazuril, totrazuril may affect all developmental stages.

Despite the importance of the various types of coccidiostats, residues thereof may remain in poultry products, including meat and eggs, which may be harmful to humans, due to cross-reactivity with human microbiome. Therefore, the use of coccidiostats is performed in the industry under tight regulations. For example, since 2006, European Council (EC) regulations allow the use of coccidiostats only for treating known infections, not as a preventative treatment. Further, the status of coccidiostats treatments is gradually changing from a food supplement to a drug. Total and final banning of coccidiostats is intended to be implemented in 2021 (Article 11 of EC Regulation 1831/2003). Such regulations are expected to be raised in many additional jurisdictions world-wide.

Accordingly, a “green”, “non-chemical” and non-hazardous feed supplement for controlling Eimeria infection is highly desirable. Particularly desirable would be a feed supplement that impairs the sporulation of the Eimeria species, since such a supplement would prevent the initial infection and therefore, may be highly efficient.

On 2017, the European Union registered an olive extract produced from Olea europaea as a botanically defined, natural product in the European Union Register of Feed Additives. On 2019, GMP+ International listed several olive-based products (olive acid oils, olive husks, olive meal, olive pulp) as GMP+ approved feed materials. Olive-related products are thus safe for animal consumption.

### SUMMARY OF THE INVENTION

The present invention provides Anti-Apicomplexan compositions, as well as methods for producing such compositions and methods for using such compositions.

The present invention is base, in part, on the surprising finding that olive mill waste, produced in high quantities during the process of producing edible olive oil from olives, may be manipulated to provide natural extracts with a potent Anti-Apicomplexan activity. The provision of compositions according to the present invention is beneficial for many reasons. For example, the methods provided here to produce beneficial compositions make use and recycle the liquid phase of olive mill, an environmentally-unfriendly and polluting material because of its high load of organic acids, acidic pH and deleterious effects on the aerobic bacteria used to detoxify wastewater in purification plants. In addition, the compositions according to the present invention may be entirely natural olive-derived-extracts, which are safe for animal growth and propagation. This advantage is of special interest as the animals, and/or their products, may be ultimately consumed by other animals, such as humans, thus preventing any risk of toxicity. Such advantages are particularly relevant in the field of commercial animal breeding and handling, as anti-coccidiosis competitors are banned from growing markets. For example, the EU has already banned antibiotics used in human medicine from being routinely added to animal feed. New Regulation completes this ban on antibiotic growth promoters in feed by prohibiting the use of the coccidiostats monensin sodium, salinomycin sodium, and avilamycin.

More, compositions according to the present invention are soluble and readily diluted in water, which makes their use highly convenient.

The present invention provides, in one aspect, an Anti-Apicomplexan composition, comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

In certain embodiments, the Anti-Apicomplexan composition comprises 3,4 Dihydroxyphenylacetic acid, Tyrosol, Caffeic acid, and Oleuropein. In certain embodiments, the Anti-Apicomplexan composition further comprises Ferulic acid. In certain embodiments, the Anti-Apicomplexan composition further comprises Hydroxytyrosol. In certain embodiments, the Anti-Apicomplexan composition further comprises p-Coumaric acid.

In certain embodiments, the Anti-Apicomplexan composition comprises Hydroxytyrosol, 3,4 Dihydroxyphenylacetic acid, Tyrosol, Caffeic acid, p-Coumaric acid, Ferulic acid and Oleuropein.

In certain embodiments, the Anti-Apicomplexan composition further comprises Tyrosol derivatives. In certain embodiments, the Anti-Apicomplexan composition further comprises Hydroxytyrosol derivatives.

In certain embodiments, the Anti-Apicomplexan composition comprises Tyrosol derivatives, Hydroxytyrosol derivatives, Hydroxytyrosol, 3,4 Dihydroxyphenylacetic acid, Tyrosol, Caffeic acid, p-Coumaric acid, Ferulic acid, and Oleuropein.

In certain embodiments, the Anti-Apicomplexan composition comprises 3,4 Dihydroxyphenylacetic acid, Tyrosol, Caffeic acid, and Oleuropein, wherein the ratio between the compounds is about 4-40 3,4 Dihydroxyphenylacetic acid to about 4-40 Tyrosol to about 4-40 Caffeic acid to about 8-80 Oleuropein.

In certain embodiments, the Anti-Apicomplexan composition comprises Ferulic acid, wherein the ratio between the compounds is about 4-40 3,4 Dihydroxyphenylacetic acid to about 4-40 Tyrosol to about 4-40 Caffeic acid to about 8-80 Oleuropein to about 2.5 to 25 Ferulic acid.

In certain embodiments, the Anti-Apicomplexan composition further comprises Hydroxytyrosol, wherein the ratio between the compounds is about 4-40 3,4 Dihydroxyphenylacetic acid to about 4-40 Tyrosol to about 4-40 Caffeic acid to about 8-80 Oleuropein to about 2-20 Hydroxytyrosol. In certain embodiments, the Anti-Apicomplexan composition further comprises p-Coumaric acid, wherein the ratio between the compounds is about 4-40 3,4 Dihydroxyphenylacetic acid to about 4-40 Tyrosol to about 4-40 Caffeic acid to about 8-80 Oleuropein to about 1.5 to 15 Coumaric acid.

In certain embodiments, the Anti-Apicomplexan composition comprises Hydroxytyrosol, 3,4 Dihydroxyphenylacetic acid, Tyrosol, Caffeic acid, p-Coumaric acid, Ferulic acid and Oleuropein, wherein the ratio between the compounds is about 2-20 Hydroxytyrosol to about 4-40 3,4 Dihydroxyphenylacetic acid to about 4-40 Tyrosol to about 4-40 Caffeic acid to about 1.5-15 p-Coumaric acid to about 2.5-25 Ferulic acid to about 8-80 Oleuropein.

In certain embodiments, the Anti-Apicomplexan composition further comprises Tyrosol derivatives, wherein the ratio between the compounds is about 4-40 3,4 Dihydroxyphenylacetic acid to about 4-40 Tyrosol to about 4-40 Caffeic acid to about 8-80 Oleuropein to about 3-30 Tyrosol derivatives. In certain embodiments, the Anti-Apicomplexan composition further comprises Hydroxytyrosol derivatives, wherein the ratio between the compounds is about 4-40 3,4 Dihydroxyphenylacetic acid to about 4-40 Tyrosol to about 4-40 Caffeic acid to about 8-80 Oleuropein to about 1-10 Hydroxytyrosol derivatives.

In certain embodiments, the Anti-Apicomplexan composition comprises Tyrosol derivatives, Hydroxytyrosol derivatives, Hydroxytyrosol, 3,4 Dihydroxyphenylacetic acid, Tyrosol, Caffeic acid, p-Coumaric acid, Ferulic acid, and Oleuropein.

The present invention provides, in another aspect, a composition comprising at least about 10 ppm of the Anti-Apicomplexan composition described above.

In certain embodiments, the composition comprises at least about 50 ppm of the Anti-Apicomplexan composition. certain embodiments, the composition comprises at least about 50 ppm, about 500 ppm, about 1000 ppm, about 2000 ppm, about 4000 ppm, about 5000 ppm or about 10,000 ppm of the Anti-Apicomplexan composition.

In certain embodiments, the composition further comprises gallic acid, caffeic acid, or vanillic acid. In certain embodiments, the composition further comprises gallic acid. In certain embodiments, the composition comprises about 50 ppm to about 550 ppm gallic acid. In certain embodiments, the composition comprises about 50 ppm to about 550 ppm gallic acid and about 2000 ppm of the Anti-Apicomplexan composition.

In certain embodiments, the composition is drinkable water or a food supplement.

In certain embodiments, the composition is an extract of olive. In certain embodiments, the composition is an extract of Olea europaea or Phillyrea latifolia.

The present invention provides, in another aspect, a method for preventing or treating an infection by Apicomplexa, comprising contacting a spore of the Apicomplexa with a composition described above.

The present invention provides, in another aspect, a method for preventing or decreasing sporulation of Apicomplexa oocytes, comprising contacting the oocytes with a composition described above.

The present invention provides, in another aspect, a method for damaging, sealing or rupturing a membrane of a cell, comprising contacting the cell with a composition described above.

In certain embodiments, the cell is an Apicomplexa cell. In certain embodiments, the Apicomplexa cell is a spore of Apicomplexa.

In certain embodiments, the Apicomplexa is Coccidia.

In certain embodiments, the Coccidia is Eimeria. In certain embodiments, the Eimeria species is selected from the group consisting of E. acervulina, E. brunetti, E. maxima, E. mitis, E. necatrix, E. praecox, E. tenella, E. adenoides, E. dispersa, E. meleagridis, E. meleagrimitis, E. gallopavonis, E. innocua, E. subrotunda, E. alabamensis, E. auburnensis, E. bovis, E. brasiliensis, E. bukidnonensis, E. canadensis, E. cylindrica, E. ellipsoidalis, E. pellita, E. subspherica, E. wyomingensis, E. zuernii, E. ahsata, E. bakuensis, E. crandallis, E. faurei, E. granulosa, E. intricata, E. marsica, E. ovinoidalis, E. pallida, E. parva, E. weybridgensis, E. alijevi, E. aspheronica, E. arloingi, E. caprina, E. caprovina, E. christenseni, E. hirci, E. jolchijevi, E. ninakohlyakimovae, E. debliecki, E. polita, E. scabra, E. spinosa, E. porci, E. neodebliecki, E. perminuta, E. suis, E. leuckarti, E. stiedae, E. flavescens, E. intestinalis, and E. macropodis.

In certain embodiments, the Coccidia is Cryptosporidium. In certain embodiments, the Cryptosporidium species is selected from the group consisting of C. andersoni, C. bailey, C. bovis, C. cervine, C. canis, C. cuniculus, C. ducismarci, C. fayeri, C. felis, C. fragile, C. galli, C. hominis, C. marcopodum, C. meleagridis, C. molnari, C. muris, C. parvum, C. ryanae, C. saurophilum, C. serpentis, C. suis, C. ubiquitum, C. viatorum, C. wrairi, and C. xiaoi.

The present invention provides, in another aspect, a method for preventing, treating or decreasing Coccidiosis incidence in a population of animals, comprising administering to the animals a composition described above.

In certain embodiments, the method comprises adding the composition to the water and/or food of the animals. In certain embodiments, the method comprises at least partly coating the surroundings of the animals with the composition.

In certain embodiments, the animals are selected from the group consisting of chickens, turkeys, cattle, sheep, goats, pigs, horses, and rabbits.

The present invention provides, in another aspect, a method for producing an Anti-Apicomplexan extract composition, comprising the steps of: obtaining olive waste, isolating the liquid phase from the olive waste, removing cellulosic compounds from the liquid phase, thereby obtaining a liquid anti-Apicomplexa extract composition, and optionally at least partly dry the liquid Anti-Apicomplexan extract composition to obtain a paste liquid Anti-Apicomplexan extract composition or a solid Anti-Apicomplexan extract composition.

In certain embodiments, the olive waste in step (i) comprises a mixture of olive mill and water. In certain embodiments, the olive mill in step (i) comprises crushed or milled olives. In certain embodiments, the olive waste in step (i) is at a temperature of about 1 to about 20° C. In certain embodiments, the olive waste in step (i) is at a temperature of about 4° C.

In certain embodiments, the liquid phase is isolated from the olive waste in step (ii) by centrifugation. In certain embodiments, the centrifugation in step (ii) comprises centrifugation at about 8000 g for about 10 minutes. In certain embodiments, the liquid phase is isolated from the olive waste in step (ii) by filtration. In certain embodiments, the filtration in step (ii) comprises filtration through a filter having a 75 μm or lower cutoff.

In certain embodiments, the cellulosic compounds in step (iii) are selected from the group consisting of cellulose, hemicellulose and lignocellulose. In certain embodiments, the cellulosic compounds in step (iii) are removed from the liquid phase by mixing the liquid phase with ethanol. In certain embodiments, the ethanol in step (iii) is 100% ethanol. In certain embodiments, the volume ratio between the liquid phase and the ethanol upon mixing is about 4:1 to 1:1. In certain embodiments, the cellulosic compounds in step (iii) are removed from the liquid phase by mixing the liquid phase with ethanol, followed by evaporation of the ethanol. In certain embodiments, the ethanol is evaporated in step (iii) by about 80 mbar vacuum at about 40° C. In certain embodiments, step (iii) is repeated until no solids precipitate from the liquid phase.

In certain embodiments, water is evaporated in step (iii) or in step (iv). In certain embodiments, water is evaporated in step (iii) or in step (iv) by about 50 mbar vacuum at about 40° C.

In certain embodiments, the liquid composition of (iii) or the paste composition of (iv) is substantially devoid of carbohydrate polymers and/or aromatic polymers. In certain embodiments, the carbohydrate polymers are selected from cellulose, hemicellulose, and a combination of both. In certain embodiments, the aromatic polymer is lignin. In certain embodiments, the liquid composition of (iii) or the paste composition of (iv) is substantially devoid of cellulose, hemicellulose, lignocellulose, lignin and/or water.

The present invention provides, in another aspect, an Anti-Apicomplexan composition, obtainable by the method described above.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### BRIEF DESCRIPTION OF THE DRAWINGS

Embodiments of the invention will be understood and appreciated more fully from the following detailed description in conjunction with the figures, which are not to scale, in which like reference numerals indicate corresponding, analogous or similar elements, and in which:

FIG. 1 presents a diagram describing an embodiment of the extraction process of the invention.

FIG. 2 presents an HPLC diagram of a liquid composition of an embodiment of the invention.

FIG. 3 presents time-dependent sporulation of Eimeria arloingi oocyst extracted from goat feces, under microscope (X100).

FIG. 4 presents a bar graph comparing the sporulation impairment (in %) of several compounds compared to control. Gallic, caffeic, and vanillic acids at the concentration of 50 mg/l inhibit 25%, 43%, and 23% of sporulation, respectively.

FIG. 5 presents dot graphs showing the sporulation (in %) of oocytes treated with different amounts of Gallic acid (FIG. 5A), different amounts of the extract of the invention (FIG. 5B), and with 2000 ppm of the extract of the invention in combination with different amounts of Gallic acid (FIG. 5C).

FIG. 6 presents a dot graph showing the Relationship between BW gain and oocyst excretion for 7 days after infection. Each point represents 20 birds.

FIG. 7 presents panels of duodenal (two upper panels), jejunal (close to Merkel diverticulum, two middle panels), and caecal lesions (two bottom panels) (score 2).

FIG. 8 presents bar graphs for the sum of duodenal, jejunal and caecal lesions (FIG. 8A), and number of organs affected (FIG. 8B) on day 14.

### DETAILED DESCRIPTION OF THE PRESENT INVENTION

Commercial production of olive oil, known for its wide-spread culinary and cosmetic use, is accompanied by the production of liquid olive mill waste. Liquid olive mill waste is considered as an environmentally-unfriendly and polluting material, since it contains high concentrations of organic acids and has an acidic pH. Such material has deleterious effects on the environment, for example on aerobic bacteria used to detoxify wastewater in purification plants.

The present invention is based in part on the finding that instead of the continued dumping of untreated liquid olive mill waste to the environment, olive mill can be manipulated to provide a beneficial and “green” product for commercial use. According to the present invention, such use is highly versatile, as the compositions of the invention, produced e.g. from liquid olive mill waste, have been found effective in several aspects. Firstly, compositions of the invention have been found to have anti-sporulation and anti-coccidiosis activities, as exemplified herein. Without being bound to any theory or mechanism, the anti-coccidiosis activity may be linked to another beneficial effect of the compositions of the invention, being the ability to deform, seal, perforate or rupture biological membranes, as also exemplified herein. In addition, despite origination, in certain embodiments, from toxic material, the compositions of the invention have been found to be safe to animals. Therefore, the compositions of the invention are highly beneficial in a variety of commercial and agricultural aspects, and even more so considering they may be produced from toxic commercial waste products.

The present invention provides, in one aspect, an Anti-Apicomplexan composition comprising Hydroxytyrosol, 3,4 Dihydroxyphenylacetic acid, Tyrosol, Caffeic acid, p-Coumaric acid, Ferulic acid, Oleuropein, or a combination thereof.

In certain embodiments, the combination comprises 2 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises 3 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises 4 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises 5 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises 6 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises Hydroxytyrosol, 3,4 Dihydroxyphenylacetic acid, Tyrosol, Caffeic acid, p-Coumaric acid, Ferulic acid and Oleuropein.

In certain embodiments, the combination comprises at least 2 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises at least 3 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises at least 4 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises at least 5 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises at least 6 different compounds selected from the group consisting of Hydroxytyrosol, 3,4 Dihydroxyphenylacetic acid, Tyrosol, Caffeic acid, p-Coumaric acid, Ferulic acid and Oleuropein. In certain embodiments, the combination comprises at least Hydroxytyrosol, 3,4 Dihydroxyphenylacetic acid, Tyrosol, Caffeic acid, p-Coumaric acid, Ferulic acid and Oleuropein.

Hydroxytyrosol is also known as 4-(2-Hydroxyethyl)-1,2-benzenediol; 3-Hydroxytyrosol; 3,4-dihydroxyphenylethanol (DOPET); Dihydroxyphenylethanol; 2-(3,4-Di-hydroxyphenyl)-ethanol (DHPE); and 3,4-dihydroxyphenolethanol (3,4-DHPEA). 3,4 Dihydroxyphenylacetic acid is also known as (3,4-Dihydroxyphenyl)acetic acid; and 2-(3,4-Dihydroxyphenyl)acetic acid. Tyrosol is also known as 4-(2-Hydroxyethyl)phenol; p-Hydroxyphenethyl alcohol; 2-(4-Hydroxyphenyl)ethanol; and 4-Hydroxyphenylethanol. Caffeic acid is also known as 3-(3,4-Dihydroxyphenyl)-2-propenoic acid; 3,4-Dihydroxy-cinnamic acid; trans-Caffeate; 3,4-Dihydroxy-trans-cinnamate; (E)-3-(3,4-dihydroxyphenyl)-2-propenoic acid; 3,4-Dihydroxybenzeneacrylicacid; 3-(3,4-Dihydroxyphenyl)-2-propenoic acid; and (2E)-3 -(3,4-Dihydroxyphenyl)prop-2-enoic acid. p-Coumaric acid is also known as (2E)-3-(4-Hydroxyphenyl)prop-2-enoic acid; (E)-3-(4-Hydroxyphenyl)-2-propenoic acid; (E)-3-(4-Hydroxyphenyl)acrylic acid; para-Coumaric acid; 4-Hydroxycinnamic acid; and β-(4-Hydroxyphenyl)acrylic acid. Ferulic acid and is also known as (E)-3-(4-hydroxy-3-methoxy-phenyl)prop-2-enoic acid; 2-propenoic acid, 3-(4-hydroxy-3-methoxyphenyl)-ferulic acid; 3-(4-hydroxy-3-methoxyphenyl)-2-propenoic acid; 3-(4-hydroxy-3-methoxyphenyl)acrylic acid; 3-methoxy-4-hydroxycinnamic acid; 4-hydroxy-3-methoxycinnamic acid; (2E)-3-(4-hydroxy-3-methoxyphenyl)-2-propenoic acid; Ferulate; Coniferic acid; trans-ferulic acid; and (E)-ferulic acid. Oleuropein is also known as (4S,5E,6S)-4-{2-[2-(3,4-dihydroxyphenyl)ethoxy]-2-oxoethyl}-5-ethylidene-6-{[(2S,3R,4S,5S,6R)-3,4,5-trihydroxy-6-(hydroxymethyl)-2-tetrahydropyranyl]oxy}-4H-pyran-3-carboxylic acid methyl ester; and 2-(3,4-Dihydroxyphenyl)ethyl [(2S,3E,4S)-3-ethylidene-2-(β-D-glucopyranosyloxy)-5-(methoxycarbonyl)-3,4-dihydro-2H-pyran-4-yl]acetate.

In certain embodiments, the composition further comprises hydroxytyrosol derivatives. In certain embodiments, the composition further comprises tyrosol derivatives. In certain embodiments, the composition further comprises hydroxytyrosol derivatives and tyrosol derivatives. The term “hydroxytyrosol derivatives” as used herein means “derivatives of hydroxytyrosol”. The term “tyrosol derivatives” as used herein means “derivatives of tyrosol”. The term “derivatives” as used herein refers to the main chemical structure of a known compound (e.g. Hydroxytyrosol or Tyrosol) with some minor differences of the substitutes on the main chemical structure or position or isomers or diastereomers of the main chemical structure of the known compound. Using HPLC analysis, compounds are identified based on internal standards where similar compounds have the same or similar retention times. In addition, Photo Diode Array (PDA) detectors are used to look at different UV wave lengths where the different compounds have different spectrum. Derivatives have a very similar PDA spectrum but with some minor differences due to differences of the substitutes, isomers or diastereomers. A comprehensive evaluation of tyrosol and hydroxytyrosol derivatives in olives is disclosed by Bartella and coworkers (Bartella, L., Mazzotti, F., Napoli, A. et al. Anal Bioanal Chem (2018) 410: 2193).

In certain embodiments, the composition comprises Hydroxytyrosol, 3,4 Dihydroxyphenylacetic acid, Tyrosol, Caffeic acid, p-Coumaric acid, Ferulic acid and/or Oleuropein, wherein the ratio between the compounds is about 0.38-38 Hydroxytyrosol or absent, to about 0.8-80 3,4 Dihydroxyphenylacetic acid or absent, to about 0.85-85 Tyrosol or absent, to about 0.85-85 Caffeic acid or absent, to about 0.34-34 p-Coumaric acid or absent, to about 0.5-50 Ferulic acid or absent, to about 1.6-160 Oleuropein or absent. Throughout this description, the term “about” is intended to cover ±10% of the specifically disclosed value. For example, “about 2” means 1.8 to 2.2.

In certain embodiments, the composition comprises Hydroxytyrosol, 3,4 Dihydroxyphenylacetic acid, Tyrosol, Caffeic acid, p-Coumaric acid, Ferulic acid and Oleuropein, wherein the ratio between the compounds is about 1-10 Hydroxytyrosol to about 2-25 3,4 Dihydroxyphenylacetic acid to about 3-25 Tyrosol to about 3-25 Caffeic acid to about 1-10 p-Coumaric acid to about 2-15 Ferulic acid to about 5-50 Oleuropein.

In certain embodiments, the composition further comprises Hydroxytyrosol derivatives and/or Tyrosol derivatives, wherein the ratio between the compounds is about 0.2-20 Hydroxytyrosol derivatives or absent, to about 0.38-38 Hydroxytyrosol or absent, to about 0.8-80 3,4 Dihydroxyphenylacetic acid or absent, to about 0.85-85 Tyrosol or absent, to about 0.66-66 Tyrosol derivatives or absent, to about 0.85-85 Caffeic acid or absent, to about 0.34-34 p-Coumaric acid or absent, to about 0.5-50 Ferulic acid or absent, to about 1.6-160 Oleuropein or absent.

In certain embodiments, the composition further comprises Hydroxytyrosol derivatives and Tyrosol derivatives, wherein the ratio between the compounds is about 1-5 Hydroxytyrosol derivatives to about 1-10 Hydroxytyrosol to about 2-25 3,4 Dihydroxyphenylacetic acid to about 3-25 Tyrosol to about 3-30 Tyrosol derivatives to about 3-25 Caffeic acid to about 1-10 p-Coumaric acid to about 2-15 Ferulic acid to about 5-50 Oleuropein.

In certain embodiments, the composition comprises Hydroxytyrosol, 3,4 Dihydroxyphenylacetic acid, Tyrosol, Caffeic acid, p-Coumaric acid, Ferulic acid and Oleuropein, and is at least as active as a control composition comprising Hydroxytyrosol, 3,4 Dihydroxyphenylacetic acid, Tyrosol, Caffeic acid, p-Coumaric acid, Ferulic acid and Oleuropein wherein the ratio between the compounds in the control composition is 3.8 Hydroxytyrosol to 8 3,4 Dihydroxyphenylacetic acid to 8.5 Tyrosol to 8.5 Caffeic acid to 3.4 p-Coumaric acid to 5 Ferulic acid to 16 Oleuropein.

In certain embodiments, the composition comprises Hydroxytyrosol derivatives, Hydroxytyrosol, 3,4 Dihydroxyphenylacetic acid, Tyrosol, Tyrosol derivatives, Caffeic acid, p-Coumaric acid, Ferulic acid and Oleuropein, and is at least as active as a control composition comprising Hydroxytyrosol derivatives, Hydroxytyrosol, 3,4 Dihydroxyphenylacetic acid, Tyrosol, Tyrosol derivatives, Caffeic acid, p-Coumaric acid, Ferulic acid and Oleuropein wherein the ratio between the compounds in the control composition is 2 Hydroxytyrosol derivatives to 3.8 Hydroxytyrosol to 8 3,4 Dihydroxyphenylacetic acid to 8.5 Tyrosol to 6.6 Tyrosol derivatives to 8.5 Caffeic acid to 3.4 p-Coumaric acid to 5 Ferulic acid to 16 Oleuropein.

In certain embodiments, the ratio between the compounds is about 3.8 Hydroxytyrosol to about 8 3,4 Dihydroxyphenylacetic acid to about 8.5 Tyrosol to about 8.5 Caffeic acid to about 3.4 p-Coumaric acid to about 5 Ferulic acid to about 16 Oleuropein.

In certain embodiments, the ratio between the compounds is about 2 Hydroxytyrosol derivatives to about 3.8 Hydroxytyrosol to about 8 3,4 Dihydroxyphenylacetic acid to about 8.5 Tyrosol to about 6.6 Tyrosol derivatives to about 8.5 Caffeic acid to about 3.4 p-Coumaric acid to about 5 Ferulic acid to about 16 Oleuropein.

In certain embodiments, the composition is substantially devoid of carbohydrate polymers and/or aromatic polymers. In certain embodiments, the carbohydrate polymers are selected from cellulose, hemicellulose, and a combination of both. In certain embodiments, the aromatic polymer is lignin.

In certain embodiments, the composition is substantially devoid of cellulose, hemicellulose, lignocellulose, lignin and/or water.

In certain embodiments, the composition is substantially devoid of carbohydrate polymers and/or aromatic polymers. In certain embodiments, the composition is substantially devoid of carbohydrate polymers. In certain embodiments, the composition is substantially devoid of aromatic polymers. In certain embodiments, the composition is substantially devoid of carbohydrate polymers and aromatic polymers. The term “substantially devoid” as used herein refers to a content of less than 5% or 50,000 ppm out of the total composition. In certain embodiments, the composition comprises less than 5% or 50,000 ppm of carbohydrate polymers and/or aromatic polymers. In certain embodiments, the composition comprises less than 1% or 10,000 ppm of carbohydrate polymers and/or aromatic polymers. In certain embodiments, the composition comprises less than 0.1% or 1,000 ppm of carbohydrate polymers and/or aromatic polymers. In certain embodiments, the carbohydrate polymers are selected from cellulose, hemicellulose, and a combination of both. In certain embodiments, the aromatic polymer is lignin. In certain embodiments, the composition is substantially devoid of cellulose, hemicellulose, lignocellulose, lignin and/or water.

The present invention further provides, in an aspect, as Anti-Apicomplexan composition, comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

In certain embodiments, the Anti-Apicomplexan composition comprises Hydroxytyrosol, 3,4 Dihydroxyphenylacetic acid, Tyrosol, and Caffeic acid. In certain embodiments, the Anti-Apicomplexan composition comprises Hydroxytyrosol, 3,4 Dihydroxyphenylacetic acid, Tyrosol, and p-Coumaric acid. In certain embodiments, the Anti-Apicomplexan composition comprises Hydroxytyrosol, 3,4 Dihydroxyphenylacetic acid, Tyrosol, and Ferulic acid. In certain embodiments, the Anti-Apicomplexan composition comprises Hydroxytyrosol, 3,4 Dihydroxyphenylacetic acid, Tyrosol, and Oleuropein.

In certain embodiments, the Anti-Apicomplexan composition comprises 3,4 Dihydroxyphenylacetic acid, Tyrosol, Caffeic acid, and p-Coumaric acid. In certain embodiments, the Anti-Apicomplexan composition comprises 3,4 Dihydroxyphenylacetic acid, Tyrosol, Caffeic acid, and Ferulic acid. In certain embodiments, the Anti-Apicomplexan composition comprises 3,4 Dihydroxyphenylacetic acid, Tyrosol, Caffeic acid, and Oleuropein.

In certain embodiments, the Anti-Apicomplexan composition comprises Hydroxytyrosol, Tyrosol, Caffeic acid, and p-Coumaric acid. In certain embodiments, the Anti-Apicomplexan composition comprises Tyrosol, Caffeic acid, p-Coumaric acid, and Ferulic acid. In certain embodiments, the Anti-Apicomplexan composition comprises Tyrosol, Caffeic acid, p-Coumaric acid, and Oleuropein.

In certain embodiments, the Anti-Apicomplexan composition comprises Hydroxytyrosol, Caffeic acid, p-Coumaric acid, and Ferulic acid. In certain embodiments, the Anti-Apicomplexan composition comprises 3,4 Dihydroxyphenylacetic acid, Caffeic acid, p-Coumaric acid, and Ferulic acid. In certain embodiments, the Anti-Apicomplexan composition comprises Caffeic acid, p-Coumaric acid, Ferulic acid, and Oleuropein.

In certain embodiments, the Anti-Apicomplexan composition comprises Hydroxytyrosol, p-Coumaric acid, Ferulic acid, and Oleuropein. In certain embodiments, the Anti-Apicomplexan composition comprises 3,4 Dihydroxyphenylacetic acid, p-Coumaric acid, Ferulic acid, and Oleuropein. In certain embodiments, the Anti-Apicomplexan composition comprises Tyrosol, p-Coumaric acid, Ferulic acid, and Oleuropein.

In certain embodiments, the Anti-Apicomplexan composition comprises Hydroxytyrosol, 3,4 Dihydroxyphenylacetic acid, Caffeic acid, and p-Coumaric acid. In certain embodiments, the Anti-Apicomplexan composition comprises Hydroxytyrosol, 3,4 Dihydroxyphenylacetic acid, Caffeic acid, and Ferulic acid. In certain embodiments, the Anti-Apicomplexan composition comprises Hydroxytyrosol, 3,4 Dihydroxyphenylacetic acid, Caffeic acid, and Oleuropein.

In certain embodiments, the Anti-Apicomplexan composition comprises Hydroxytyrosol, 3,4 Dihydroxyphenylacetic acid, p-Coumaric acid, and Ferulic acid. In certain embodiments, the Anti-Apicomplexan composition comprises Hydroxytyrosol, 3,4 Dihydroxyphenylacetic acid, p-Coumaric acid, and Oleuropein.

In certain embodiments, the Anti-Apicomplexan composition comprises Hydroxytyrosol, 3,4 Dihydroxyphenylacetic acid, Ferulic acid, and Oleuropein.

In certain embodiments, the Anti-Apicomplexan composition comprises Hydroxytyrosol, Tyrosol, Caffeic acid, and Ferulic acid. In certain embodiments, the Anti-Apicomplexan composition comprises Hydroxytyrosol, Tyrosol, Caffeic acid, and Oleuropein.

In certain embodiments, the Anti-Apicomplexan composition comprises Hydroxytyrosol, Caffeic acid, p-Coumaric acid, and Oleuropein.

In certain embodiments, the Anti-Apicomplexan composition comprises Hydroxytyrosol, Tyrosol, Ferulic acid, and Oleuropein. In certain embodiments, the Anti-Apicomplexan composition comprises Hydroxytyrosol, Caffeic acid, Ferulic acid, and Oleuropein.

In certain embodiments, the Anti-Apicomplexan composition comprises 3,4 Dihydroxyphenylacetic acid, Tyrosol, p-Coumaric acid, and Ferulic acid. In certain embodiments, the Anti-Apicomplexan composition comprises 3,4 Dihydroxyphenylacetic acid, Tyrosol, p-Coumaric acid, and Oleuropein.

In certain embodiments, the Anti-Apicomplexan composition comprises 3,4 Dihydroxyphenylacetic acid, Caffeic acid, p-Coumaric acid, and Oleuropein.

In certain embodiments, the Anti-Apicomplexan composition comprises 3,4 Dihydroxyphenylacetic acid, Caffeic acid, Ferulic acid, and Oleuropein.

In certain embodiments, the Anti-Apicomplexan composition comprises Tyrosol, Caffeic acid, Ferulic acid, and Oleuropein.

In certain embodiments, the Anti-Apicomplexan composition comprises Caffeic acid, p-Coumaric acid, Ferulic acid, and Oleuropein.

In certain embodiments, the Anti-Apicomplexan composition comprises Hydroxytyrosol, p-Coumaric acid, Ferulic acid, and Oleuropein. In certain embodiments, the Anti-Apicomplexan composition comprises Tyrosol, p-Coumaric acid, Ferulic acid, and Oleuropein.

In certain embodiments, the Anti-Apicomplexan composition further comprises Ferulic acid. In certain embodiments, the Anti-Apicomplexan composition further comprises Hydroxytyrosol. In certain embodiments, the Anti-Apicomplexan composition further comprises p-Coumaric acid. In certain embodiments, the Anti-Apicomplexan composition further comprises Ferulic acid and Hydroxytyrosol. In certain embodiments, the Anti-Apicomplexan composition further comprises Hydroxytyrosol and p-Coumaric acid. In certain embodiments, the Anti-Apicomplexan composition further comprises Ferulic acid and p-Coumaric acid.

In certain embodiments, the Anti-Apicomplexan composition comprises Hydroxytyrosol, 3,4 Dihydroxyphenylacetic acid, Tyrosol, Caffeic acid, p-Coumaric acid, Ferulic acid and Oleuropein.

In certain embodiments, the Anti-Apicomplexan composition further comprises Tyrosol derivatives. In certain embodiments, the Anti-Apicomplexan composition further comprises Hydroxytyrosol derivatives.

In certain embodiments, the Anti-Apicomplexan composition comprises Tyrosol derivatives, Hydroxytyrosol derivatives, Hydroxytyrosol, 3,4 Dihydroxyphenylacetic acid, Tyrosol, Caffeic acid, p-Coumaric acid, Ferulic acid, and Oleuropein.

In certain embodiments, the Anti-Apicomplexan composition comprises 3,4 Dihydroxyphenylacetic acid, Tyrosol, Caffeic acid, and Oleuropein, wherein the ratio between the compounds is about 4-40 3,4 Dihydroxyphenylacetic acid to about 4-40 Tyrosol to about 4-40 Caffeic acid to about 8-80 Oleuropein.

In certain embodiments, the Anti-Apicomplexan composition comprises 3,4 Dihydroxyphenylacetic acid, Tyrosol, Caffeic acid, and Oleuropein, wherein the ratio between the compounds is about 6-24 3,4 Dihydroxyphenylacetic acid to about 7-28 Tyrosol to about 7-28 Caffeic acid to about 13-52 Oleuropein.

In certain embodiments, the Anti-Apicomplexan composition comprises 3,4 Dihydroxyphenylacetic acid, Tyrosol, Caffeic acid, and Oleuropein, wherein the ratio between the compounds is about 13 3,4 Dihydroxyphenylacetic acid to about 14 Tyrosol to about 14 Caffeic acid to about 26 Oleuropein.

In certain embodiments, the Anti-Apicomplexan composition further comprises Ferulic acid, wherein the ratio between the compounds is about 4-40 3,4 Dihydroxyphenylacetic acid to about 4-40 Tyrosol to about 4-40 Caffeic acid to about 8-80 Oleuropein to about 2.5 to 25 Ferulic acid.

In certain embodiments, the Anti-Apicomplexan composition further comprises Ferulic acid, wherein the ratio between the compounds is about 6-24 3,4 Dihydroxyphenylacetic acid to about 7-28 Tyrosol to about 7-28 Caffeic acid to about 13-52 Oleuropein to about 4 to 16 Ferulic acid.

In certain embodiments, the Anti-Apicomplexan composition further comprises Ferulic acid, wherein the ratio between the compounds is about 13 3,4 Dihydroxyphenylacetic acid to about 14 Tyrosol to about 14 Caffeic acid to about 26 Oleuropein to about 8 Ferulic acid.

In certain embodiments, the Anti-Apicomplexan composition further comprises Hydroxytyrosol, wherein the ratio between the compounds is about 4-40 3,4 Dihydroxyphenylacetic acid to about 4-40 Tyrosol to about 4-40 Caffeic acid to about 8-80 Oleuropein to about 2-20 Hydroxytyrosol.

In certain embodiments, the Anti-Apicomplexan composition further comprises Hydroxytyrosol, wherein the ratio between the compounds is about 6-24 3,4 Dihydroxyphenylacetic acid to about 7-28 Tyrosol to about 7-28 Caffeic acid to about 13-52 Oleuropein to about 3-12 Hydroxytyrosol.

In certain embodiments, the Anti-Apicomplexan composition further comprises Hydroxytyrosol, wherein the ratio between the compounds is about 13 3,4 Dihydroxyphenylacetic acid to about 14 Tyrosol to about 14 Caffeic acid to about 26 Oleuropein to about 6 Hydroxytyrosol.

In certain embodiments, the Anti-Apicomplexan composition further comprises p-Coumaric acid, wherein the ratio between the compounds is about 4-40 3,4 Dihydroxyphenylacetic acid to about 4-40 Tyrosol to about 4-40 Caffeic acid to about 8-80 Oleuropein to about 1.5 to 15 p-Coumaric acid.

In certain embodiments, the Anti-Apicomplexan composition further comprises p-Coumaric acid, wherein the ratio between the compounds is about 6-24 3,4 Dihydroxyphenylacetic acid to about 7-28 Tyrosol to about 7-28 Caffeic acid to about 13-52 Oleuropein to about 3 to 12 p-Coumaric acid.

In certain embodiments, the Anti-Apicomplexan composition further comprises p-Coumaric acid, wherein the ratio between the compounds is about 13 3,4 Dihydroxyphenylacetic acid to about 14 Tyrosol to about 14 Caffeic acid to about 26 Oleuropein to about 6 p-Coumaric acid.

In certain embodiments, the Anti-Apicomplexan composition comprises Hydroxytyrosol, 3,4 Dihydroxyphenylacetic acid, Tyrosol, Caffeic acid, p-Coumaric acid, Ferulic acid and Oleuropein, wherein the ratio between the compounds is about 2-20 Hydroxytyrosol to about 4-40 3,4 Dihydroxyphenylacetic acid to about 4-40 Tyrosol to about 4-40 Caffeic acid to about 1.5-15 p-Coumaric acid to about 2.5-25 Ferulic acid to about 8-80 Oleuropein.

In certain embodiments, the Anti-Apicomplexan composition further comprises Tyrosol derivatives, wherein the ratio between the compounds is about 4-40 3,4 Dihydroxyphenylacetic acid to about 4-40 Tyrosol to about 4-40 Caffeic acid to about 8-80 Oleuropein to about 3-30 Tyrosol derivatives.

In certain embodiments, the Anti-Apicomplexan composition further comprises Hydroxytyrosol derivatives, wherein the ratio between the compounds is about 4-40 3,4 Dihydroxyphenylacetic acid to about 4-40 Tyrosol to about 4-40 Caffeic acid to about 8-80 Oleuropein to about 1-10 Hydroxytyrosol derivatives.

In certain embodiments, the Anti-Apicomplexan composition comprises Tyrosol derivatives, Hydroxytyrosol derivatives, Hydroxytyrosol, 3,4 Dihydroxyphenylacetic acid, Tyrosol, Caffeic acid, p-Coumaric acid, Ferulic acid, and Oleuropein.

In certain embodiments, the ratio between the compounds in the Anti-Apicomplexan composition is about 3.8 Hydroxytyrosol to about 8 3,4 Dihydroxyphenylacetic acid to about 8.5 Tyrosol to about 8.5 Caffeic acid to about 3.4 p-Coumaric acid to about 5 Ferulic acid to about 16 Oleuropein.

In certain embodiments, the ratio between the compounds in the Anti-Apicomplexan composition is about 2 Hydroxytyrosol derivatives to about 3.8 Hydroxytyrosol to about 8 3,4 Dihydroxyphenylacetic acid to about 8.5 Tyrosol to about 6.6 Tyrosol derivatives to about 8.5 Caffeic acid to about 3.4 p-Coumaric acid to about 5 Ferulic acid to about 16 Oleuropein.

The present invention further provides, in another aspect, a composition comprising at least about 10 ppm of the Anti-Apicomplexan composition described above.

The term “at least 10 ppm” as use herein refers e.g. to any composition consisting essentially of the Anti-Apicomplexan composition and therefore comprising million ppm of the Anti-Apicomplexan composition, as well as to any composition in which the Anti-Apicomplexan composition is mixed with or diluted by any other material, as long as the components of the Anti-Apicomplexan composition are at least found in a quantity or concentration of 100 ppm. The term specifically relates to any composition consisting essentially of the Anti-Apicomplexan composition (million ppm), or comprising the Anti-Apicomplexan composition diluted up to 10,000 fold (100 ppm).

In certain embodiments, the composition comprises up to 1,000,000 ppm of the Anti-Apicomplexan composition. In certain embodiments, the composition comprises up to 100,000 ppm of the Anti-Apicomplexan composition. In certain embodiments, the composition comprises up to 10,000 ppm of the Anti-Apicomplexan composition. In certain embodiments, the composition comprises up to 1,000 ppm of the Anti-Apicomplexan composition.

In certain embodiments, the composition comprises at least 100,000 ppm to up to 1,000,000 ppm of the Anti-Apicomplexan composition. In certain embodiments, the composition comprises at least 10,000 ppm to up to 100,000 ppm of the Anti-Apicomplexan composition. In certain embodiments, the composition comprises at least 1,000 ppm to up to 10,000 ppm of the Anti-Apicomplexan composition. In certain embodiments, the composition comprises at least 100 ppm to up to 1,000 ppm of the Anti-Apicomplexan composition.

In certain embodiments, the composition comprises at least 100 ppm, at least 1,000 ppm, or at least 10,000 ppm, to up to 10,000 ppm, up to 100,000 ppm, or up to 1,000,000 ppm of the Anti-Apicomplexan composition.

As the Anti-Apicomplexan composition may be too concentrated or potent to allow easy and safe use, there may rise a need to dilute the Anti-Apicomplexan before use. Thus, a relatively diluted Anti-Apicomplexan composition is herein referred to strictly as a “composition”, which comprises at least about 10 ppm of the Anti-Apicomplexan composition described above.

In certain embodiments, the composition comprises at least about 50 ppm of the Anti-Apicomplexan composition. In certain embodiments, the composition comprises at least about 500 ppm of the Anti-Apicomplexan composition. In certain embodiments, the composition comprises at least about 1000 ppm of the Anti-Apicomplexan composition. In certain embodiments, the composition comprises at least about 2000 ppm of the Anti-Apicomplexan composition. In certain embodiments, the composition comprises at least about 3000 ppm of the Anti-Apicomplexan composition. In certain embodiments, the composition comprises at least about 4000 ppm of the Anti-Apicomplexan composition. In certain embodiments, the composition comprises at least about 5000 ppm of the Anti-Apicomplexan composition. In certain embodiments, the composition comprises at least about 6000 ppm of the Anti-Apicomplexan composition. In certain embodiments, the composition comprises at least about 7000 ppm of the Anti-Apicomplexan composition. In certain embodiments, the composition comprises at least about 8000 ppm of the Anti-Apicomplexan composition. In certain embodiments, the composition comprises at least about 9000 ppm of the Anti-Apicomplexan composition. In certain embodiments, the composition comprises at least about 10000 ppm of the Anti-Apicomplexan composition.

The composition of claim 19, comprising about 50 ppm, 500 ppm, 1000 ppm, 2000 ppm, 4000 ppm, 5000 ppm or 10000 ppm of the Anti-Apicomplexan composition.

In certain embodiments, the composition further comprises gallic acid, caffeic acid, or vanillic acid. In certain embodiments, the composition further comprises gallic acid. In certain embodiments, the composition further comprises caffeic acid. As person skilled in the art would understand, the phrase “a composition comprising the Anti-Apicomplexan composition and caffeic acid” means that the composition comprises both caffeic acid originated in the Anti-Apicomplexan composition and caffeic acid which is not originated in the Anti-Apicomplexan composition, but added separately. In certain embodiments, the composition further comprises vanillic acid.

In certain embodiments, the composition comprises about 50 ppm to about 550 ppm gallic acid. In certain embodiments, the composition comprises about 100 ppm gallic acid. In certain embodiments, the composition comprises about 150 ppm gallic acid. In certain embodiments, the composition comprises about 200 ppm gallic acid. In certain embodiments, the composition comprises about 250 ppm gallic acid. In certain embodiments, the composition comprises about 300 ppm gallic acid. In certain embodiments, the composition comprises about 350 ppm gallic acid. In certain embodiments, the composition comprises about 400 ppm gallic acid. In certain embodiments, the composition comprises about 450 ppm gallic acid. In certain embodiments, the composition comprises about 500 ppm gallic acid. In certain embodiments, the composition comprises about 550 ppm gallic acid.

In certain embodiments, the composition comprises about 50 ppm to about 550 ppm gallic acid and about 2000 ppm of the Anti-Apicomplexan composition.

In certain embodiments, the composition comprises drinkable water or a food supplement. In certain embodiments, the composition comprises drinkable water. In certain embodiments, the composition comprises a food supplement.

In certain embodiments, the composition is formulated as drinkable water or as a food supplement. In certain embodiments, the composition is formulated as drinkable water. In certain embodiments, the composition is formulated as a food supplement.

In certain embodiments, the composition is an olive extract. In certain embodiments, the composition is an Olea europaea extract. In certain embodiments, the composition is a Phillyrea latifolia extract. As person skilled in the art would know, there are several well-known techniques to identify a composition as an extract, e.g. as a botanical extract. For example, DNA from the composition can be recovered, sequenced, and readily searched in on-line tools, such as the NIH Blast search engine.

In certain embodiments, the composition comprises at least 200 ppm of the Anti-Apicomplexan composition. In certain embodiments, the composition comprises at least 300 ppm of the Anti-Apicomplexan composition. In certain embodiments, the composition comprises at least 400 ppm of the Anti-Apicomplexan composition. In certain embodiments, the composition comprises at least 500 ppm of the Anti-Apicomplexan composition. In certain embodiments, the composition comprises at least 1000 ppm of the Anti-Apicomplexan composition. In certain embodiments, the composition comprises at least 1500 ppm of the Anti-Apicomplexan composition. In certain embodiments, the composition comprises at least 2000 ppm of the Anti-Apicomplexan composition. In certain embodiments, the composition comprises at least 2500 ppm of the Anti-Apicomplexan composition. In certain embodiments, the composition comprises at least 3000 ppm of the Anti-Apicomplexan composition. In certain embodiments, the composition comprises at least 3500 ppm of the Anti-Apicomplexan composition. In certain embodiments, the composition comprises at least 4000 ppm of the Anti-Apicomplexan composition.

In certain embodiments, the composition comprises at least 2000 ppm of the anti-Apicomplexa composition and further comprises at least 400 ppm Gallic acid.

In certain embodiments, the composition comprises at least 4000 ppm of the anti-Apicomplexa composition and further comprising at least 250 ppm Gallic acid.

In certain embodiments, the composition comprises at least 8000 ppm of the anti-Apicomplexa composition and further comprising at least 150 ppm Gallic acid.

In certain embodiments, the composition comprises at least 16000 ppm of the anti-Apicomplexa composition and further comprising at least 100 ppm Gallic acid.

In certain embodiments, the composition comprises at least 5000 ppm or 10000 ppm of the anti-Apicomplexa composition and at least 0 ppm or 500 ppm Gallic acid. In certain embodiments, the composition comprises at least 10000 ppm of the anti-Apicomplexa composition and no Gallic acid. In certain embodiments, the composition comprises at least 10000 ppm of the anti-Apicomplexa composition and at least 500 ppm Gallic acid. In certain embodiments, the composition comprises at least 5000 ppm of the anti-Apicomplexa composition and no Gallic acid. In certain embodiments, the composition comprises at least 5000 ppm of the anti-Apicomplexa composition and at least 500 ppm Gallic acid.

In certain embodiments, the composition comprises 5000 ppm to 10000 ppm of the anti-Apicomplexa composition and 0 ppm to 500 ppm Gallic acid. In certain embodiments, the composition comprises 10000 ppm of the anti-Apicomplexa composition and no Gallic acid. In certain embodiments, the composition comprises 10000 ppm of the anti-Apicomplexa composition and 500 ppm Gallic acid. In certain embodiments, the composition comprises 5000 ppm of the anti-Apicomplexa composition and no Gallic acid. In certain embodiments, the composition comprises 5000 ppm of the anti- Apicomplexa composition and 500 ppm Gallic acid.

According to some embodiments, the composition may be administered to the water provided to poultry. According to some embodiments, the composition is administered to the poultry via the water provided thereto on a regular basis, whether or not the poultry is known to be infected with at least one member of the Eimeria species. The Eimeria species encompasses all the Eimeria genus, e.g., in poultry, E. acervulina. E. necatrix. E. tenella.

According to some embodiments, the composition is in the form of a powder. According to some embodiments, the composition is in the form of a solution. According to some embodiments, when the composition is in the form of a solution, the solvent is water.

According to some embodiments, the composition comprises about 10.3 mg hydroxytyrosol per gram. According to some embodiments, the composition comprises about 8.2 mg 3,4 dihydroxyphenylacetic acid per gram. According to some embodiments, the composition comprises about 15.6 mg tyrosol per gram. According to some embodiments, the composition comprises about 9 mg caffeic acid per gram. According to some embodiments, the composition comprises about 0.82 mg vanillic acid per gram. According to some embodiments, the composition comprises about 3.4 mg p-coumaric acid per gram. According to some embodiments, the composition comprises about 6.6 mg ferulic acid per gram. According to some embodiments, the composition comprises about 24 mg oleuropein per gram.

According to some embodiments, the composition comprises between 5.3 and 16.7 mg hydroxytyrosol per gram. According to some embodiments, the composition comprises between 8.0 and 8.6 mg 3,4 dihydroxyphenylacetic acid per gram. According to some embodiments, the composition comprises about 8.2 mg 3,4 dihydroxyphenylacetic acid per gram. According to some embodiments, the composition comprises between 10.6 and 21.0 mg tyrosol per gram. According to some embodiments, the composition comprises between 8.4 and 10.0 mg caffeic acid per gram. According to some embodiments, the composition comprises 2.5 to 4.7 mg p-coumaric acid per gram. According to some embodiments, the composition comprises 4.0 to 9.5 mg ferulic acid per gram. According to some embodiments, the composition comprises 14 to 43 mg oleuropein per gram.

According to some embodiments, the composition is enriched with about 5-15% gallic acid. According to some embodiments, the gallic acid is obtained from a natural source. According to some embodiments, the composition only comprises materials obtained from natural sources.

The present invention further provides, in another aspect, a method for producing an Anti-Apicomplexan extract composition, comprising the steps of: (i) obtaining olive waste, (ii) isolating a liquid phase from the olive waste, (iii) removing cellulosic compounds from the liquid phase, thereby obtaining a liquid Anti-Apicomplexan extract composition, and optionally (iv) at least partly dry the liquid Anti-Apicomplexan extract composition to obtain a paste Anti-Apicomplexan extract composition or a solid Anti-Apicomplexan extract composition.

In certain embodiments, the olive is Olea europaea or Phillyrea latifolia. In certain embodiments, the olive is Olea europaea. In certain embodiments, the olive is Phillyrea latifolia.

The term “liquid composition” as used herein refers to a composition being in a liquid state or formulated as a liquid at room temperature, e.g. at 20° C. to 30° C. The term “paste composition” as used herein refers to a composition being in a semi-solid state or formulated as a semi-solid at room temperature. The term “solid composition” as used herein refers to a composition being in a solid state or formulated as a solid at room temperature.

In certain embodiments, the olive waste in step (i) comprises a mixture of olive mill and water. In certain embodiments, the olive mill in step (i) comprises crushed or milled olives. In certain embodiments, the olive waste in step (i) is at a temperature of about 1 to about 20° C. In certain embodiments, the olive waste in step (i) is at a temperature of about 4° C.

In certain embodiments, the liquid phase is isolated from the olive waste in step (ii) by centrifugation. In certain embodiments, the centrifugation in step (ii) comprises centrifugation at about 8000 g for about 10 minutes. In certain embodiments, the liquid phase is isolated from the olive waste in step (ii) by filtration. In certain embodiments, the filtration in step (ii) comprises filtration through a filter having a 75 μm or lower cutoff In certain embodiments, the cellulosic compounds in step (iii) are selected from the group consisting of cellulose, hemicellulose and lignocellulose.

In certain embodiments, the cellulosic compounds in step (iii) are removed from the liquid phase by mixing the liquid phase with ethanol. In certain embodiments, the ethanol in step (iii) is 100% ethanol. In certain embodiments, the volume ratio between the liquid phase and the ethanol upon mixing is about 10:1 to 1:1. In certain embodiments, the volume ratio between the liquid phase and the ethanol upon mixing is about 4:1 to 1:1. In certain embodiments, the volume ratio between the liquid phase and the ethanol upon mixing is about 4:1 to 1:1.cellulosic compounds in step (iii) are removed from the liquid phase by mixing the liquid phase with ethanol, followed by evaporation of the ethanol. In certain embodiments, the ethanol is evaporated in step (iii) by about 80 mbar vacuum at about 40° C. In certain embodiments, the step (iii) is repeated until no solids precipitate from the liquid phase.

In certain embodiments, the water is evaporated in step (iii) or in step (iv). In certain embodiments, the water is evaporated in step (iii) or in step (iv) by about 50 mbar vacuum at about 40° C.

In certain embodiments, the liquid composition of (iii) or the paste composition or solid composition of (iv) is substantially devoid of carbohydrate polymers and/or aromatic polymers. In certain embodiments, the liquid composition of (iii) or the paste composition or solid composition of (iv) is substantially devoid of carbohydrate polymers. In certain embodiments, the liquid composition of (iii) or the paste composition or solid composition of (iv) is substantially devoid of aromatic polymers. In certain embodiments, the liquid composition of (iii) or the paste composition or solid composition of (iv) is substantially devoid of carbohydrate polymers and aromatic polymers. The term “substantially devoid” as used herein refers to a content of less than 5% or 50,000 ppm out of the total composition. In certain embodiments, the composition comprises less than 5% or 50,000 ppm of carbohydrate polymers and/or aromatic polymers. In certain embodiments, the composition comprises less than 1% or 10,000 ppm of carbohydrate polymers and/or aromatic polymers. In certain embodiments, the composition comprises less than 0.1% or 1,000 ppm of carbohydrate polymers and/or aromatic polymers. In certain embodiments, the carbohydrate polymers are selected from cellulose, hemicellulose, and a combination of both. In certain embodiments, the aromatic polymer is lignin. In certain embodiments, the liquid composition of (iii) or the paste composition or solid composition of (iv) is substantially devoid of cellulose, hemicellulose, lignocellulose, lignin and/or water.

It would be understood to any person of skill in the art that part or parts of an entity may be alternatively referred to as percentages (%) from the entity or as parts-per-million (ppm) from the entity. For example, a half of an entity may be referred to as 50% of the entity and/or as 500,000 ppm from the entity.

According to some embodiments, during the method of production, the liquid phase may be stored at a temperature of about 0° C. to about 10° C. at any step before performing the next step. According to some embodiments, during the method of production, the liquid phase may be stored at a temperature of about 4° C. at any step before performing the next step.

According to some embodiments, during the method of production, the liquid phase is not warmed to a temperature exceeding 50° C. According to some embodiments, during the method of production, the liquid phase is not warmed to a temperature exceeding 45° C. According to some embodiments, during the method of production, the liquid phase is not warmed to a temperature exceeding 40° C.

According to some embodiments, during the method of production, any of the steps may be repeated any number of times, as required. According to some embodiments, during the method of production, any sequence of steps may be repeated any number of times.

The present invention further provides, in another aspect, an Anti-Apicomplexan liquid composition or an Anti-Apicomplexan paste composition or an Anti-Apicomplexan solid composition, obtainable or obtained by the method described above.

The present invention further provides, in another aspect, a method for deforming, coating, sealing, damaging or rupturing a membrane of a cell, comprising contacting the cell with a composition described above.

The present invention further provides, in another aspect, a method for preventing or treating an infection by Apicomplexa, comprising contacting the Apicomplexa with a composition described above.

The present invention further provides, in another aspect, a method for preventing or treating an infection by Apicomplexa, comprising contacting a spore of the Apicomplexa with a composition described above.

The present invention further provides, in another aspect, a method for preventing or decreasing sporulation of Apicomplexa oocytes, comprising contacting the oocytes with a composition described above.

The present invention further provides, in another aspect, a method for damaging, sealing or rupturing a membrane of a cell, comprising contacting the cell with a composition described above.

In certain embodiments, the cell is a cell of Apicomplexa. In certain embodiments, the Apicomplexa cell is a spore of Apicomplexa. In certain embodiments, the Apicomplexa is Coccidia. In certain embodiments, the Coccidia is Eimeria.

In certain embodiments, the Eimeria species is selected from the group consisting of E. acervuhna, E. brunetti, E. maxima, E. mitis, E. necatrix, E. praecox, E. tenella, E. adenoides, E. dispersa, E. meleagridis, E. meleagrimitis, E. gallopavonis, E. innocua, E. subrotunda, E. alabamensis, E. auburnensis, E. bovis, E. brasihensis, E. bukidnonensis, E. canadensis, E. cylindrica, E. ellipsoidahs, E. pellita, E. subspherica, E. wyomingensis, E. zuernii, E. ahsata, E. bakuensis, E. crandallis, E. faurei, E. granulosa, E. intricata, E. marsica, E. ovinoidalis, E. pallida, E. parva, E. weybridgensis, E. alijevi, E. aspheronica, E. arloingi, E. caprina, E. caprovina, E. christenseni, E. hirci, E. jolchijevi, E. ninakohlyakimovae, E. debliecki, E. polita, E. scabra, E. spinosa, E. porci, E. neodebliecki, E. perminuta, E. suis, E. leuckarti, E. stiedae, E. flavescens, E. intestinalis and E. macropodis.

In certain embodiments, the Eimeria is Eimeria spp.

In certain embodiments, the Coccidia is Cryptosporidium.

In certain embodiments, the Cryptosporidium species is selected from the group consisting of C. andersoni, C. bailey, C. bovis, C. cervine, C. canis, C. cuniculus, C. ducismarci, C. fayeri, C. felis, C. fragile, C. galli, C. hominis, C. marcopodum, C. meleagridis, C. molnari, C. muris, C. parvum, C. ryanae, C. saurophilum, C. serpentis, C. suis, C. ubiquitum, C. viatorum, C. wrairi, and C. xiaoi.

The present invention further provides, in another aspect, a method for preventing, treating or decreasing Coccidiosis incidence in a population of animals, comprising administering to the animals a composition described above.

In certain embodiments, the method comprises adding the composition to the water and/or food of the animals. In certain embodiments, the method comprises adding the composition to the water of the animals. In certain embodiments, the method comprises adding the composition to the food of the animals. In certain embodiments, the method comprises at least partly coating the surroundings of the animals with the composition.

In certain embodiments, the animals are selected from the group consisting of chickens, turkeys, cattle, sheep, goats, pigs, horses, and rabbits. In certain embodiments, the animals are chickens. In certain embodiments, the animals are turkeys.

In certain embodiments, the Apicomplexa is of the Phylum Apicomplexa. In certain embodiments, the Phylum Apicomplexa is of the Class Conoidasida. In certain embodiments, the Class Conoidasida is of the Subclass Coccidia. In certain embodiments, the Subclass Coccidia is of the Order Eucoccidiorida. In certain embodiments, the Order Eucoccidiorida is of the Suborder Eimeriorina. In certain embodiments, the Suborder Eimeriorina is of the Families of Eimeriidae or Cryptosporidiidae. In certain embodiments, the Suborder Eimeriorina is of the Family of Eimeriidae. In certain embodiments, the Suborder Eimeriorina is of the Family of Cryptosporidiidae. In certain embodiments, the Family of Eimeriidae is of the Genus Eimeria. In certain embodiments, the Family of Cryptosporidiidae is of the Genus Cryptosporidium.

Unless explicitly stated, the method embodiments described herein are not constrained to a particular order or sequence. Additionally, some of the described method embodiments or elements thereof can occur or be performed simultaneously, at the same point in time, or concurrently.

Various embodiments have been presented. Each of these embodiments may of course include features from other embodiments presented, and embodiments not specifically described may include various features described herein.

### EXAMPLES

### Example 1. Preparation of an Olive Waste Extract

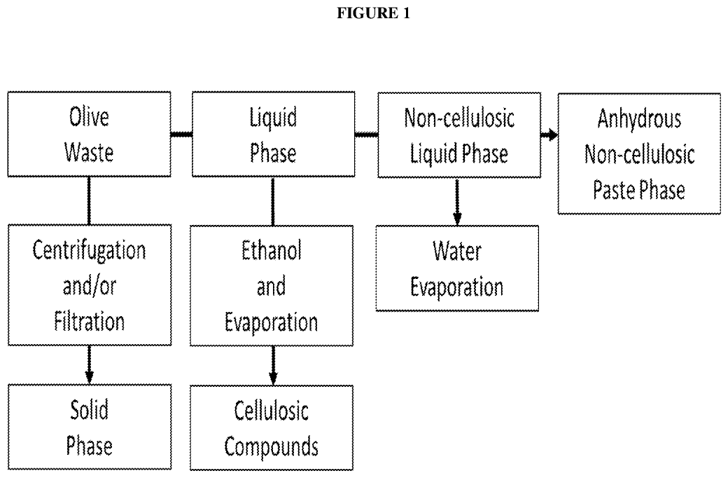

Olive are harvested and conveyed to the olive mill, crushed, added water, and decantation and centrifugation are used to separate the three phases: solid, olive mill with water and oil. The olive mill plus water is considered olive waste and used to produce the “Antisolv extract” or the “Antisolv extract composition” of the invention. One liter of olive waste stored at 4° C., was centrifuged (8000 g) for 10 minutes, and then the liquid phase was subjected to filtration using a 75 μm sieve. Then, the liquid phase was centrifuged and an additional amount of 250 ml of 100% ethanol was added. Then the liquid was concentrated under high vacuum (starting 1000 mbar till 80 mbar) using a rotor-evaporator at 40° C. until it reached a volume of 250 ml, in order to remove the ethanol. Then, in order to remove the water, the same procedure is used under a pressure of 50 mbars. The process of evaporation and addition of 100% ethanol was repeated until no more solid (which is a cellulosic mixture) precipitated from the liquid phase. The liquid phase, which mainly contains a polyphenolic/flavonoid mixture, was evaporated under high vacuum to produce 40 grams of a dark-brown paste, referred to herein as “AntiSolv extract”, “Antosolv”, “olive waste extract” or “extract”, interchangeably. A diagram presenting an embodiment of the extraction process of the invention is presented in FIG. 1.

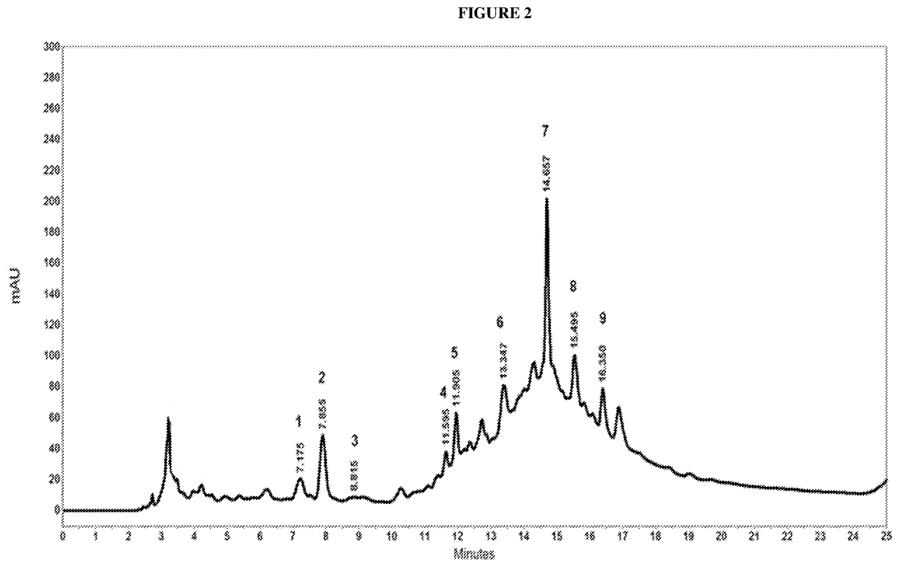

FIG. 2 presents an HPLC diagram of a sample of the extract. Dry sample of the extract was dissolved in 100% methanol or 1:1 (v:v) ethanol 100% and isonitrile, then injected into the HPLC for analysis. Peaks #1-2 represent hydroxytyrosol (including derivatives), peak #3 represents 3,4 dihydroxyphenylacetic acid, peaks #4-5 represent tyrosol (including derivatives), peak #6 represents caffeic acid, peak #7 represents p-coumaric acid, peak #8 represents ferulic acid, and peak #9 represents oleuropein (Table 1).

It is noted that a Folin reaction (the reaction of amino acids in alkaline solution with 1,2-naphthoquinone-4-sulfonate (Folin reagent) was used to test the phenolic content of the obtained extract. It is estimated that the phenolics contained in Antisolv are the active agents involved in the inhibition of sporulation, as they bond with proteins in membranes, impairing permeability. The concentration of the main compounds in the obtained extract, as detailed in FIG. 2, are presented in Table 1 below:

TABLE 1
Polyphenol concentrations in a sample of Antisolv extract (61.8 ppm in total).

| Peak | | | | |
|---|---|---|---|---|
| number | Phenol | AUC* | Concentration | % of total |
| 1 | Hydroxytyrosol Derivatives | 50050 | 2 ppm | 3.2 |
| 2 | Hydroxytyrosol | 173452 | 3.8 ppm | 6.1 |
| 3 | 3,4 Dihydroxyphenylacetic acid | 16960 | 8 ppm | 12.9 |
| 4 | Tyrosol | 289537 | 8.5 ppm | 13.7 |
| 5 | Tyrosol Derivatives | 231114 | 6.6 ppm | 10.6 |
| 6 | Caffeic acid | 636073 | 8.5 ppm | 13.7 |
| 7 | p-Coumaric acid | 1525346 | 3.4 ppm | 5.5 |
| 8 | Ferulic acid | 1156378 | 5 ppm | 8.1 |
| 9 | Oleuropein | 420757 | 16 ppm | 25.8 |

*Area under the curve (arbitrary units).

### Example 2. Efficacy of an Olive Waste Extract In-Vitro

Eimeria-infected feces were collected from goats and stored at 4° C. Later, 3 grams of feces were mixed with 42 ml of tap water. The sample was homogenized and passed through a mesh, after which the filtered liquid was collected into 50 ml tubes. The liquid was then washed three times with cold tap water and centrifuged (1,700 G, 5 minutes, 20° C.). In each wash, the supernatant was carefully removed and approximately 30 to 40 ml of cold water was added. After the last wash, the water was removed and replaced with saturated sugar flotation solution (500 gr sucrose in 320 ml double distilled water), wherein each tube was filled with up to 45 ml of the sugar flotation solution. Each tube was centrifuged twice, wherein after each round of centrifugation, the ring-shaped phase formed on the top of the sugar solution was collected (about 1-2 ml collected after each round) and preserved in a 50 ml plastic tube.

The plastic tube was filled with cold water and centrifuged again. Oocysts were found in the sediment after centrifugation. The water was gently removed and the concentration of the oocysts was evaluated. The oocysts were then stored at 4° C. until use (the use of unsporulated oocysts that are older than two weeks was avoided). It is noted that a 2% potassium dichromate solution may be used for conservation. The unsporulated oocytes were washed in cold phosphate buffered saline (PBS, X10) before use in order to remove potassium dichromate (if used). The concentration of the oocysts was adjusted to approximately 30,000/ml.

Extract was prepared according to Example 1 above. Extract can be mixed with Phosphate buffer saline (PBS, X1, w:w, 1:10) if not perfectly soluble in water.

1 ml of Antisolv extract at concentrations of 500, 1000, 2000, and 4,000 ppm together with 1 ml of the oocysts solution were placed in 5 ml glass tubes, wherein each glass tube was covered with Parafilm®, with holes to allow for oxygen supply and vortexed at 150 rpm. Each assay was in triplicates for each concentration of extract and gallic acid purchased at Sigma. In addition, six concentrations of gallic acid (50, 150, 250, 350, 450, 550 ppm) were added to 2000 and 4000 ppm of the invention extract to check synergism of gallic acid and the extract on impairment of sporulation. The glass tubes were covered, with holes for air, stored at room temperature (26-28° C.) for 48 hours on a rotator. Control glass tubes (3 per assay), filled with 1 ml of oocyst solution and 1 ml DDH₂O (doubly-distilled water) or PBS (Phosphate buffered saline) without extract was prepared.

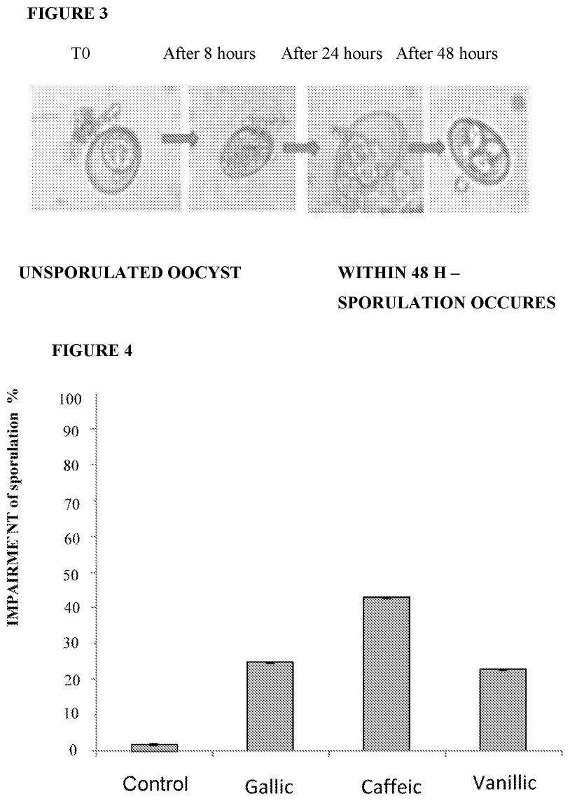

After incubation, the tubes were washed with PBS and kept at 4° C. until counting the oocysts. The assay was repeated for six replications (six concentrations, five droplets each, each having at least 100 oocysts per droplet, 30 samples in total) and sporulation percentage was averaged within extract concentration. Sporulation was identified by microscopy, as shown in FIG. 3, and sporulation rate was calculated.

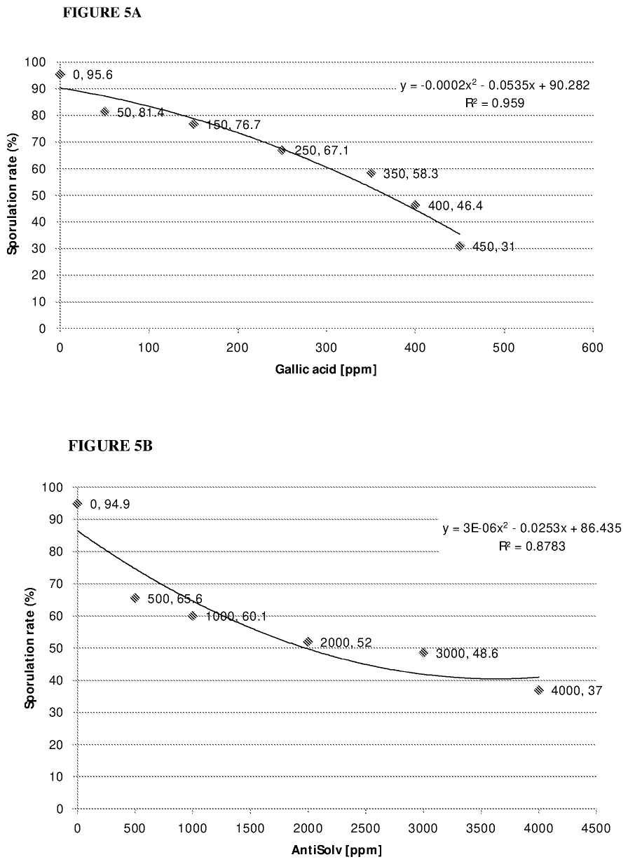

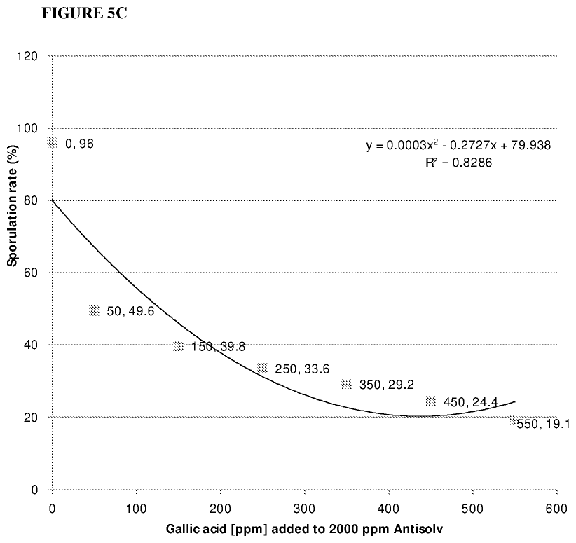

There was no impairment of sporulation in the controls (containing water only), compared, for example to 50 ppm of gallic, caffeic, and vanillic acid (FIG. 4), which are produced during the degradation of polyphenols in the intestine, or the extract of the invention (FIG. 5A-5C). Gallic acid was selected because it is an accepted and well-regulated nutrition supplement for poultry.

Sporulation was impaired by the extract of the invention in a dose-dependent fashion (FIG. 5A), reaching 60% inhibition of sporulation at the maximal concentration of 4,000 ppm of Antisolv extract, encompassing all its components.

Gallic acid increased the impairment of sporulation when given as supplement to the extract of the invention: as presented in FIG. 5B, the addition of gallic acid to 2000 ppm extract reduced the sporulation percentage of the oocysts to about 30%. As presented in FIG. 5C, the addition of gallic acid to 4000 ppm extract reduced the sporulation percentage of the oocysts to about 20%.

### Example 3. Efficacy and Safety of an Olive Waste Extract In-Vivo

Chicks aged 2-4 weeks were allowed ad-libitum drinking strictly from water containing 0.5% (5000 ppm) of Antisolv extract of the invention. It was found that these chicks could thrive well, and their growth was not affected, compared to chicks drinking untreated tap water.

### Example 4. Efficacy of an Olive Waste Extract In-Vivo

A dose-response experiment was conducted: treatments are 0, 2000, 4000, and 8000 ppm of extract with/without 400 ppm gallic acid. Chicks are uninfected or infected with 50,000-100,000 oocysts of mixed Eimeria (Table 2).

Five replicates of 20 chicks for treatment (800 chicks). Water and feed intake weekly, feed efficiency throughout; morbidity, opg (oocyst count per gram droppings), mortality—are all monitored. Liver and intestine morphology (location and severity of lesions) are examined in ten chicks/treatment. Feed intake and growth performance are recorded, stats by two-way ANOVA, repeated measurements. Water fluidity and pH, weekly; tube-clogging events are monitored.

### Example 5. Efficacy of an Olive Waste Extract In-Vivo

160 chicks allotted to 8 treatments, encompassing two groups of uninfected birds and six groups of Eimeria-infected birds, were tested. Birds were administered water treatment from 7 days of age and infected when 10-days old.

Inoculation was individually, per os, by using a dose of Paracox 5, a live attenuated anticoccidial vaccine including administered at the twenty-fold the recommended dose (Table 2), in a volume of 1 ml, when chicks were 10 days old. Uninfected birds were given the same volume of saline. Eimeria spp were as in Table 2.

TABLE 2
The composition of inoculated Eimeria spp.

| Eimeria spp. | Per dose | Per bird |
|---|---|---|
| E. acervulina HP | 500 per dose | 10.000 |
| E. brunetti HP | 100 per dose | 2,000 |
| E. maxima CP | 200 per dose | 4,000 |
| E. maxima MEP | 100 per dose | 2,000 |
| E. mitis HP | 1000 per dose | 20,000 |
| E. necatrix HP | 500 per dose | 10,000 |
| E. praecox HP | 100 per dose | 2,000 |
| E. tenella HP | 500 per dose | 1,000 |

Water was supplied in 3-liter poultry troughs that were re-filled when needed. Tap water was initially given and water supplementation was initiated three days after chicks' arrival. Water treatments for the uninfected (C) birds were no antisolvent and no gallic acid (C_AS0%_GA0) and 1% antisolvent with 500 ppm of gallic acid (C_AS1%_GA500).

Water treatments for the infected (T) birds were bi-factorial with 3 levels of antisolvent (AS; 0, 0.5, and 1%) and two levels of gallic acid (GA; 0 and 500 ppm) resulting in 6 groups: T_AS0%_GA1, T_AS0.5%_GA0, T_AS1%_GA0, T_AS0.5%_GA0, T_AS0.5% _GA500, and T_AS1%_GA500.

Birds were wing-tagged and weighed on infection day and then, 7 days after infection. Half of the birds were weighed and humanely euthanized by exposure to CO₂ on day 14 after infection and the second half on day 17 after infection. Water intake was recorded. Droppings were collected from a 1 m² paper sheet placed in each group 12 h before each weighing and kept at 4° C. until oocysts were counted under a grated microscope at ×100 and ×400 at days 0, and 7.

Liver was inspected and spleen was inspected and all parts of the gastro-intestinal tract, encompassing the duodenum, ileum-jejunum, and caeca were noted for lesions in a 0-4 scale. No existing method, based on mono-species Eimeria infection, was satisfactory. The duodenum was examined at three sites (proximal, medial, distal) and a separate score was recorded for each. In contrast, only one score was recorded for the ileum-jejunum, and one for the caeca. Organ scores were termed D (average of 3 sites along the duodenum), J, and C. Three composite scores were established: a. sum of the average duodenal score, the jejunal score and the caecal score; b. sum of the maximal duodenal, jejunal and caecal scores; c. number of lesion-affected organs (1-3).

Data for body weight (BW) and average daily gain was log-transformed in order to homogenize variances. The C_AS0%_GA0 and C_AS0%_GA0 were compared in order to assess the effect of infection. The C_AS0%_GA0 and the C_AS1%_GA500 were compared to evaluate possible toxicity due to water treatment. All groups encompassing bi-factorial levels of AS and GA were compared by bi-factorial ANOVA.

For the analysis of lesions, for each treatment and slaughter day, the Wilcoxon Rank Sum Test was run. Then, using the Iman-Connover method, a rank transformation was done on original data and a two-way ANOVA was run on the ranked data. The treatment x day interaction was significant for J. Sum DJC, and #organs affected and Pairwise comparisons were made by Tukey test.

Results.

A total of 6 chicks died throughout experiment, independent of treatment, two of them after inoculation, including one in the C_AS0%_GA0 uninfected group.

Water intake was little affected, if any, by water supplements (Table 3). No signs of toxicity whatsoever were apparent in chicks drinking AS and/or GA-supplemented water.

TABLE 3
Water intake in uninfected chicks given no water supplement
(C_AS0%_GA0) or water with 1% antisolvent
and 500 ppm gallic acid (C_AS1%_GA500), or
infected and given water with bi-factorial
combinations of antisolvent (AS, 0, 0.5%, and 1%,
corresponding to 0 ppm, 5,000 ppm, and 10,000 ppm,
respectively) and gallic acid (0 and 500 ppm)

| | Water intake |
|---|---|
| Treatment | (ml chick⁻¹ dr⁻¹) |
| C_AS0%_GA0 | 119 |
| T_AS0.5%_GA0 | 150 |
| T_AS1%_GA0 | 144 |
| T_AS0%_GA500 | 132 |
| T_AS0.5%_GA500 | 121 |
| T_AS1%_GA500 | 127 |
| T_AS0%_GA0 | 121 |
| C_AS1%_GA500 | 137 |

Unvaccinated medicated control birds substantially did not excrete oocysts for 14 days after infection, indicating freedom from both between-pen contamination by the vaccine and invasion of the chicken-house by extraneous coccidial infection (Table 4). Birds treated with combinations of antisolvent and gallic acid had very low excretion of oocysts, much lower than counterparts given plain water (5,200-14,200 in treated-infected, compared with 96,000 in non-treated-infected birds). However, non-treated fowls (T_AS0%_GA0) did not excrete oocysts at day 14, probably because of higher immune response.

TABLE 4
Oocyst counts in droppings (gr⁻¹), days are relative to infection day.

| | d 0 | d 7 | d 14 |
|---|---|---|---|
| C_AS1%_GA500 | 0 | 0 | 320 |
| C_AS0%_GA0 | 0 | 400 | 200 |
| T_AS1%_GA0 | 0 | 5,200 | 1,000 |
| T_AS0%_GA500 | 0 | 10,650 | 3,600 |
| T_AS1%_GA500 | 0 | 11,800 | 0 |
| T_AS0.5%_GA0 | 0 | 14,200 | 0 |
| T_AS0.5%_GA500 | 0 | 22,400 | 200 |
| T_AS0%_GA0 | 0 | 96,000 | 0 |

Infection resulted in significantly lower body weight from d 7 throughout experiment (Table 5).

TABLE 5
Body weight of chicks throughout experiment,
days are relative to infection day (Means ± SE).

| | d 0 | d 7 | d 14 | D 17 |
|---|---|---|---|---|
| C_AS0%_GA0* | 168.3 ± 4.1 | 430.2 ± 16.6 A | 1048.0 ± 45.5 A | 1418.8 ± 80.4 A |
| C_AS1%_GA500 | 164.1 ± 5.8 | 375.5 ± 20.1 | 6 ± 51.5 | 1261.4 ± 97.0 |
| T_AS0%_GA0*, ** | 159.0 ± 4.2 | 357.4 ± 13.4 B bc | 914.9 ± 39.7 B bc | 1183.0 ± 79.2 B bcd |
| T_AS0%_GA500** | 168.9 ± 4.3 | 399.5 ± 13.4 a | 984.4 ± 40.1 ab | 1290.0 ± 83.4 ab |
| T_AS0.5%_GA0** | 169.5 ± 4.5 | 408.8 ± 14.5 a | 1049.8 ± 43.7 a | 1427.5 ± 51.5 a |
| T_AS0.5%_GA500** | 175.6 ± 4.3 | 398.5 ± 14.3 ab | 994.6 ± 43.7 ab | 1289.0 ± 80.0 abc |
| T_AS1%_GA0** | 175.8 ± 5.2 | 406.5 ± 19.2 a | 964.4 ± 56.4 c | 1284.4 ± 102.4 abc |
| T_AS1%_GA500** | 156.8 ± 5.5 | 357.6 ± 17.4 c | 879.9 ± 40.5 c | 1201.1 ± 70.1 cd |
| p*** | P = 0.22 | P = 0.04 | P = 0.05 | P = 0.07 |

*Column-wise, different uppercase letters denote statistically significant differences between C_AS0%_GA0 and T_AS0%_GA0, i.e., effect of infection.
**Column-wise, within T groups, values with same lowercase letters do not differ statistically.
***Column-wise, P is for the comparison of water treatments within infected chicks.

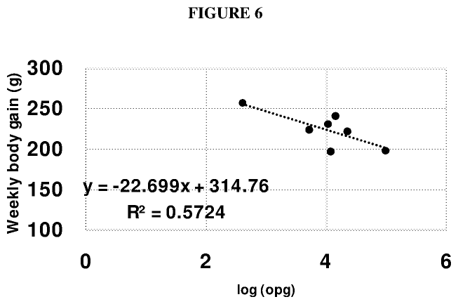

The effect of infection on average daily gain (ADG, Table 6) was particularly deleterious on days 0-7 (P<0.001), in which, group-wise, when log (opg) was plotted against BW gain for the first 7 d after infection, a significant negative effect was shown (FIG. 6). The BW of uninfected, untreated birds and infected birds given the AS0.5% GA0 treatment was identical throughout. In other words, drinking water with AS0.5% (5,000 ppm Antisolve) annihilated the effect of infection on growth performance.

TABLE 6
Average daily gain (g d⁻¹) of chicks throughout experiment.

| | Days 0-7 | Days 8-14 | Days 14-17 |
|---|---|---|---|
| C_AS0%_GA0 | 37.4 ± 2.1 | 56.2 ± 2.7 | 103 ± 7.2 |
| C_AS1%_GA500 | 30.2 ± 2.4 | 53.3 ± 3.0 | 70.5 ± 4.8 |
| T_AS0%_GA0 | 28.3 ± 1.4 | 50.7 ± 2.5 | 84.2 ± 7.7 |
| T_AS0%_GA500 | 32.9 ± 1.4 | 53.2 ± 2.5 | 99.9 ± 6.2 |
| T_AS0.5%_GA0 | 34.2 ± 1.5 | 58.3 ± 2.7 | 94.8 ± 5.6 |
| T_AS0.5%_GA500 | 31.8 ± 1.5 | 54.2 ± 2.8 | 84.4 ± 6.8 |
| T_AS1%_GA0 | 33.0 ± 2.2 | 50.7 ± 3.6 | 93.0 ± 9.7 |
| T_AS1%_GA500 | 28.7 ± 1.8 | 47.5 ± 2.2 | 83.7 ± 6.2 |
| Within T birds | AS0.5 > AS0 = AS1 | AS0.5 > AS0 > AS1 | AS0 > AS0.5 = AS1 |

The performance growth of infected birds with no water treatment (T_AS0%_GA0) or with the maximal treatment (T_AS1%_GA500) was inferior, compared to all other treatments. There was no advantage in adding gallic acid if an AS treatment was given. Providing infected birds with AS0.5%_GA0 was the best treatment but that treatment did not result in statistically higher BW, compared with T_AS0%_GA500, T_AS0.5%_GA0, T_AS0.5%_GA500, T_AS1%_GA0 at the end of experiment. For average daily gains within infected birds (Table 6), water treatments ranked AS0.5>AS1=AS0, AS0.5>AS1=AS0, AS0>AS0.5=AS1 for days 0-7, 8-14, 14-17, respectively. Only for the three last days, antisolvent supplementation had no effect on growth performance.

Overall, data shows that infection with Eimeria impaired growth, providing water with 0.5% AS helped alleviating the effect of infection, GA was not needed if AS was provided and the combination of AS1% and GA500 was deleterious to growth performance.

No events of liver lesions, enlargement or discoloration were found. Some birds presented an enlarged or whitish spleen, without relation to infection or treatments. However, non-infected birds in the C_AS0%_GA0 had the least frequency of bloody spleen.

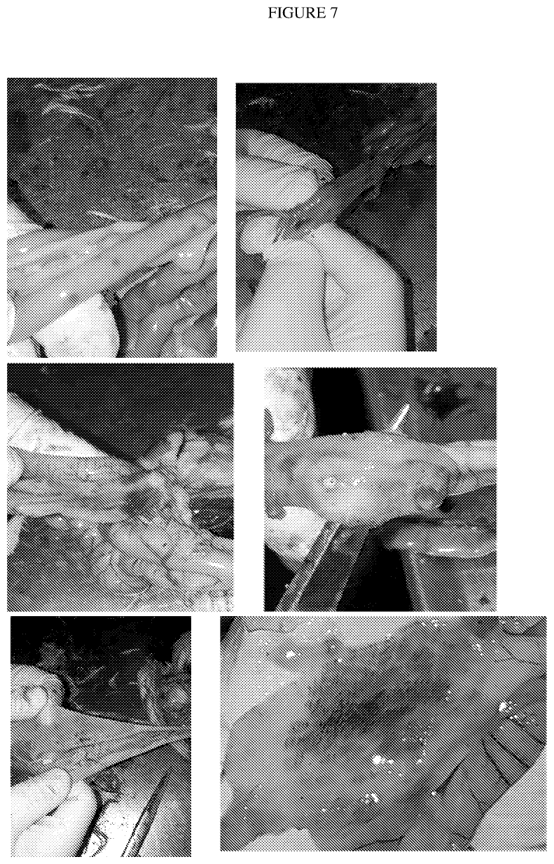

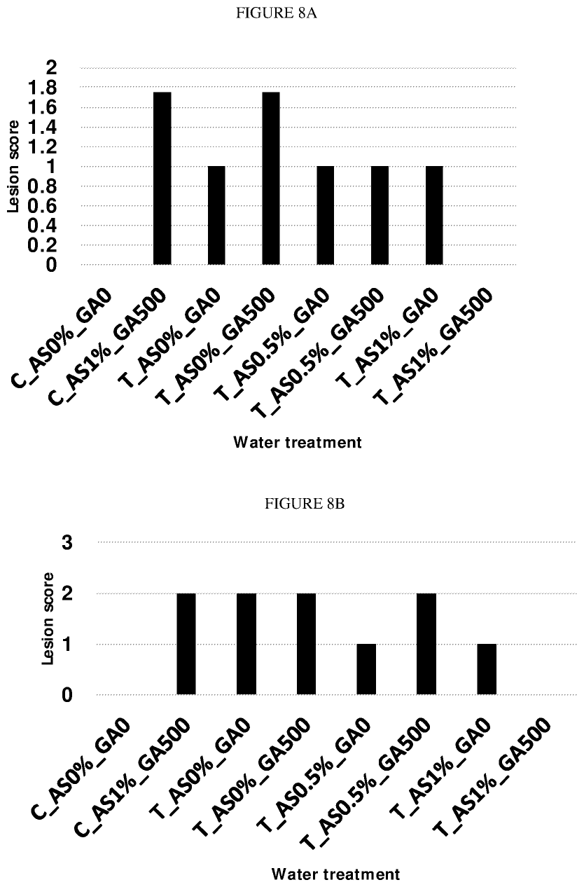

The infection was effective, as evidenced by lesions along the GIT (FIG. 7). Overall, more lesions were found on day 17 than on day 14. The non-infected C_AS0%_GA0 birds had no lesions on day 14 and a very milk infection on day 17 and it was the least-affected group throughout. No lesions were found in this group on day 14 after infection but the number of lesion-afflicted birds increased from day 14 to 17 (FIG. 8), evidence of oocyst recycling from infected to non-infected birds during the removal of fowls on d 14. Lesions were noted more frequently in the duodenum and jejunum, both in the serosa and mucosa, than in caeca. Failure to detect lesions in T_AS1%_GA500 birds is challenged by the oocysts count in their droppings on day 7 post-infection. For both the sum of lesions and number of organs affected, the groups ranked C_AS0%_GA0<C_AS1%_GA500=T_AS0.5%_GA500, the rest of groups showing intermediate values.

The results above demonstrate that (a) providing broilers with a water-dispersible AS that inhibits sporulation, with or without gallic acid, would attenuate coccidiosis damages in poultry; (b) AS in water did not affect water consumption; (c) infection was effective, as monitored by opg in droppings, and impaired growth, albeit lesion scores were low; and (d) providing water with 0.5% AS cancelled the deleterious effect of Eimeria infection on growth. Gallic acid was not needed if AS was provided and the combination of AS1% and GA500 was even deleterious to growth performance.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An Anti-Apicomplexan composition, comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

2. The composition of claim 1, comprising:
a. 3,4 Dihydroxyphenylacetic acid, Tyrosol, Caffeic acid, and Oleuropein;
b. 3,4 Dihydroxyphenylacetic acid, Tyrosol, Caffeic acid, Oleuropein, and Ferulic acid;
c. 3,4 Dihydroxyphenylacetic acid, Tyrosol, Caffeic acid, Oleuropein, Ferulic acid and Hydroxytyrosol;
d. 3,4 Dihydroxyphenylacetic acid, Tyrosol, Caffeic acid, Oleuropein and Hydroxytyrosol;
e. 3,4 Dihydroxyphenylacetic acid, Tyrosol, Caffeic acid, Oleuropein and p-Coumaric acid:
f. 3,4 Dihydroxyphenylacetic acid, Tyrosol, Caffeic acid, Oleuropein, Ferulic acid and p-Coumaric acid;
g. 3,4 Dihydroxyphenylacetic acid, Tyrosol, Caffeic acid, Oleuropein, Ferulic acid, Hydroxytyrosol and p-Coumaric acid; or
h. 3,4 Dihydroxyphenylacetic acid, Tyrosol, Caffeic acid, Oleuropein Hydroxytyrosol and p-Coumaric acid.

3.-6. (canceled)

7. The composition of claim 1, further comprising Tyrosol derivatives, Hydroxytyrosol derivatives, or both Tyrosol derivatives and Hydroxytyrosol derivatives.

8.-9. (canceled)

10. The composition of claim 1, comprising 3,4 Dihydroxyphenylacetic acid, Tyrosol, Caffeic acid, and Oleuropein, wherein the ratio between the compounds is about 4-40 3,4 Dihydroxyphenylacetic acid to about 4-40 Tyrosol to about 4-40 Caffeic acid to about 8-80 Oleuropein.

11. The composition of claim 10, further comprising at least one of:
a. Ferulic acid, at a ratio between about 2.5 to 25 Ferulic acid;
b. Hydroxytyrosol, at a ratio between about 2-20 Hydroxytyrosol;
c. p-Coumaric acid, at a ratio between about 1.5 to 15 p-Coumaric acid;
d. Tyrosol derivatives, at a ratio between about 3-30 Tyrosol derivatives; and
e. Hydroxytyrosol derivatives, at a ratio between about 1-10 Hydroxytyrosol derivatives.

12.-17. (canceled)

18. A composition comprising at least about 10 ppm, 50 ppm, 500 ppm, 1000 ppm, 2000 ppm, 4000 ppm, 5000 ppm or 10,000 ppm of an Anti-Apicomplexan composition comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

19. (canceled)

20. The composition of claim 18, comprising about 50 ppm, 500 ppm, 1000 ppm, 2000 ppm, 4000 ppm, 5000 ppm or 10,000 ppm of the Anti-Apicomplexan composition.

21. The composition of claim 18, further comprising gallic acid, caffeic acid, or vanillic acid.

22. (canceled)

23. (canceled)

24. The composition of claim 18, further comprising about 50 ppm to about 550 ppm gallic acid and about 2000 ppm of the Anti-Apicomplexan composition.

25. The composition of claim 18, being drinkable water or a food supplement.

26. (canceled)

27. A method for preventing or treating an infection by Apicomplexa, comprising contacting a spore of the Apicomplexa with a composition comprising at least about 10 ppm, 50 ppm, 500 ppm, 1000 ppm, 2000 ppm, 4000 ppm, 5000 ppm or 10,000 ppm of an Anti-Apicomplexan composition comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

28.-31. (canceled)

32. The method of claim 27, wherein the Apicomplexa is Coccidia, wherein:
a. the Coccidia is Eimeria, and wherein the Eimeria species is selected from the group consisting of E. acervulina, E. brunetti, E. maxima, E. mitis, E. necatrix, E. praecox, E. tenella, E. adenoides, E. dispersa, E. meleagridis, E. meleagrimitis, E. gallopavonis, E. innocua, E. subrotunda, E. alabamensis, E. auburnensis, E. bovis, E. brasiliensis, E. bukidnonensis, E. canadensis, E. cylindrica, E. ellipsoidalis, E. pellita, E. subspherica, E. wyomingensis, E. zuernii, E. ahsata, E. bakuensis, E. crandallis, E. faurei, E. granulosa, E. intricata, E. marsica, E. ovinoidalis, E. pallida, E. parva, E. weybridgensis, E. alijevi, E. aspheronica, E. arloingi, E. caprina, E. caprovina, E. christenseni, E. hirci, E. jolchijevi, E. ninakohlyakimovae, E. debliecki, E. polita, E. scabra, E. spinosa, E. porci, E. neodebliecki, E. perminuta, E. suis, E. leuckarti, E. stiedae, E. flavescens, E. intestinalis, and E. macropodis, or
b. the Coccidia is Cryptosporidium, and the Cryptosporidium species is selected from the group consisting of C. andersoni, C. bailey, C. bovis, C. cervine, C. canis, C. cuniculus, C. ducismarci, C. fayeri, C. felis, C. fragile, C. galli, C. hominis, C. marcopodum, C. meleagridis, C. molnari, C. muris, C. parvum, C. ryanae, C. saurophilum, C. serpentis, C. suis, C. ubiquitum, C. viatorum, C. wrairi, and C. xiaoi.

33.-36. (canceled)

37. A method for preventing, treating or decreasing Coccidiosis incidence in a population of animals, comprising administering to the animals a composition comprising at least about 10 ppm, 50 ppm, 500 ppm, 1000 ppm, 2000 ppm, 4000 ppm, 5000 ppm or 10,000 ppm of an Anti-Apicomplexan composition comprising a combination of at least four compounds selected from the group consisting of: Hydroxytyrosol, 3,4 Dihydroxyphenylacetic acid, Tyrosol, Caffeic acid, p-Coumaric acid, Ferulic acid, and Oleuropein.

38. The method of claim 37, comprising adding the composition to the water and/or food of the animals, or at least partly coating the surroundings of the animals with the composition, wherein the animals are selected from the group consisting of chickens, turkeys, cattle, sheep, goats, pigs, horses, and rabbits.

39.-40. (canceled)

41. A method for producing an Anti-Apicomplexan extract composition, comprising the steps of:
(i) obtaining olive waste,
(ii) isolating the liquid phase from the olive waste,
(iii) removing cellulosic compounds from the liquid phase, thereby obtaining a liquid anti-Apicomplexa extract composition, and optionally
(iv) at least partly dry the liquid Anti-Apicomplexan extract composition to obtain a paste Anti-Apicomplexan extract composition or a solid Anti-Apicomplexan extract composition.

42. The method of claim 41, wherein the olive waste in step (i):
a. comprises a mixture of olive mill and water;
b. comprises crushed or milled olives;
c. is at a temperature of about 1 to about 20° C.;
d. is at a temperature of about 4° C.; or
e. any combination of the above.

43.-45. (canceled)

46. The method of claim 41, wherein the liquid phase is isolated from the olive waste in step (ii) by centrifugation or filtration; wherein the centrifugation in step (ii) comprises centrifugation at about 8000 g for about 10 minutes; and wherein the filtration in step (ii) comprises filtration through a filter having a 75 μm or lower cutoff.

47-49. (canceled)

50. The method of claim 41, wherein:
a. the cellulosic compounds in step (iii) are selected from the group consisting of cellulose, hemicellulose and lignocellulose;
b. the cellulosic compounds in step (iii) are removed from the liquid phase by mixing the liquid phase with ethanol, wherein the ethanol is 100% ethanol, and wherein the volume ratio between the liquid phase and the ethanol upon mixing is about 4:1 to 1:1;
c. the cellulosic compounds in step (iii) are removed from the liquid phase by mixing the liquid phase with ethanol, followed by evaporation of the ethanol, and wherein the ethanol is evaporated by about 80 mbar vacuum at about 40° C.;
d. step (iii) is repeated until no solids precipitate from the liquid phase;
e. water is evaporated in step (iii) or in step (iv), and wherein water is evaporated by about 50 mbar vacuum at about 40° C.;
f. the liquid composition of (iii) or the paste composition of (iv) is substantially devoid of carbohydrate polymers and/or aromatic polymers, wherein the carbohydrate polymers are selected from cellulose, hemicellulose, and a combination of both, and wherein the aromatic polymer is lignin; or
g. any combination of the above.

51.-63. (canceled)
